SEQUENCE LISTING (12 sequences)

SEQ ID NO: 1 - protein, 550 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGSPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNDPEATNALIDKDGWLHSGDIAYWDEDEHFFIVDRLKSLIKYKGCQVAPAELESILLQHPNIFDAGVAGLPGDDAGELPAAVVVLEHGKTMTEKEIVDYVASQVTTAKKLRGGVVFVDEVPKGLTGKLDARKIREILIKAKKGGKSKL

SEQ ID NO: 2 - protein, 171 aa, Artificial Sequence
  Amino acid sequence of a synthetic luciferase commercially named “NanoLuc” (Promega); derived from Oplophorus gracilirostris
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 3 - protein, 148 aa, Artificial Sequence
  Amino acid sequence of a synthetic luciferase commercially named “TurboLuc” (Thermo Scientific); derived from the marine copepod Metridia pacifica luciferase family
MEAEAERGKLPGKKLPLEVLIELEANARKAGCTRGCLICLSKIKCTAKMKKYIPGRCADYGGDKKTGQAGIVGAIVDIPEISGFKEMEPMEQFIAQVDRCADCTTGCLKGLANVKCSDLLKKWLPGRCATFADKIQSEVDNIKGLAGD

SEQ ID NO: 4 - protein, 209 aa, Artificial Sequence
  Amino acid sequence of a synthetic luciferase commercially named “Lucia” (InvivoGen); derived from planktonic copepods
MEIKVLFALICIAVAEAKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDR

SEQ ID NO: 5 - protein, 736 aa, Artificial Sequence
  Amino acid sequence of an AAV capsid designated “S3”
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 6 - protein, 736 aa, Artificial Sequence
  Recombinant AAV capsid protein, SEQ ID NO 31 from WO 2013/029030
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 7 - protein, 736 aa, Artificial Sequence; identical to SEQ ID NO: 6
  Recombinant AAV capsid protein, SEQ ID NO 46 from WO 2013/029030

SEQ ID NO: 8 - protein, 736 aa, Artificial Sequence; identical to SEQ ID NO: 6
  Recombinant AAV capsid protein, SEQ ID NO 47 from WO 2013/029030

SEQ ID NO: 9 - protein, 738 aa, Artificial Sequence
  Recombinant AAV capsid protein, SEQ ID NO 54 from WO 2013/029030
MAADGYLPDWLEDNLSEGIREWWDLKPGAPKPKANQQKQDDGRGLVLPGYKYLGPFNGLDKGEPVNAADATALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTIQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSNNVEFAVNTEGVYSEPRPIGTRYLTRNL

SEQ ID NO: 10 - protein, 738 aa, Artificial Sequence
  Recombinant AAV capsid protein, SEQ ID NO 56 from WO 2013/029030
MAADGYLPDWLEDTLSEGIRQWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTIQVFTDSEYQLPYVLGSAHQGCLPPFPADVFMIPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYYKSNNVEFAVNTEGVYSEPRPIGTRYLTRNL

SEQ ID NO: 11 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 12 - protein, 959 aa, Streptococcus pyogenes
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

(19) United States
(12) Patent Application Publication
(10) Pub. No.: US 2023/0093697 A1
(43) Pub. Date: Mar. 23, 2023

(54) IMPROVED ASSAY FOR DETERMINING NEUTRALISING ANTIBODY TITRE TO A VIRAL VECTOR

(71) Applicant: Freeline Therapeutics Limited, Stevenage (GB)

(72) Inventors: Jonathan FOLEY, London (GB); Erald SHEHU, London (GB); Allison DANE, London (GB)

(21) Appl. No.: 17/795,767

(22) PCT Filed: Jan. 28, 2021
(86) PCT No.: PCT/GB2021/050198

§ 371 Date: Jul. 27, 2022

(30) Foreign Application Priority Data
Jan. 28, 2020 (GB) 2001203.5
Feb. 4, 2020 (GB) 2001496.5
May 12, 2020 (GB) 2006987.8

(51) Int. Cl. G01N 33/68; C12Q 1/6897; C12Q 1/66

(52) U.S. Cl. CPC G01N 33/6854; C12Q 1/6897; C12Q 1/66; G01N 2333/165

(57) ABSTRACT

The present invention relates to an improved assay and in particular to an improved assay that is capable of consistently measuring antibody titre, especially neutralising antibody (NAb) titre, at lower thresholds and/or with greater speed than conventionally-known assays. The invention further relates to use of such assays in combination with the provision of gene therapy and/or in combination with the provision of methods aimed at removal/depletion of neutralising antibodies from a patient.

### FIELD OF THE INVENTION

The present invention relates to an improved assay and in particular to an improved assay that is capable of consistently measuring antibody titre, especially neutralising antibody (NAb) titre, at lower thresholds and/or with greater speed than conventionally-known assays. The invention further relates to use of such assays in combination with the provision of gene therapy and/or in combination with the provision of methods aimed at removal/depletion of neutralising antibodies from a patient.

### BACKGROUND TO THE INVENTION

Gene therapy using viruses (such as adeno-associated virus (AAV) and lentiviruses) is increasingly acknowledged as having potential as a therapeutic platform for treatment of many rare diseases including haemophilia A and B as well as a range of lysosomal disorders. There are currently multiple trials seeking to correct these disorders using AAV gene therapy (Doshi and Arruda 2018, Smith, et al. 2013). Despite this promising approach, humoral immunity against viral vectors is an obstacle to gene therapy since it leads to clearance of the vector from a patient's system before the vector has had the opportunity to facilitate transduction of the transgene of interest. In particular, antibodies having specificity for the AAV capsid are highly prevalent in humans, and are known to cross-react with a wide range of AAV serotypes (there is a high degree of homology of capsid protein sequence across the different serotypes). Pre-existing neutralising antibodies (NAbs) to AAV have been shown to modulate the efficacy of AAV vector mediated gene therapy by blocking vector transduction or by redirecting distribution of AAV vectors to tissues other than the target organ (Calcedo and Wilson, Humoral Immune Response to AAV. 2013). Moreover, NAbs can be present in 30-70% of the general population for a given serotype (Calcedo, Morizono, et al. 2011, Li, et al. 2012). Even relatively low titres of neutralising antibodies are thought to be able to block AAV transduction (Kruzik, et al. 2019). Together, the prevalence of NAbs to AAV and the biological relevance of low titres reduce the number of patients eligible for new treatments. This is particularly the case where a patient has already been exposed to e.g. wild-type AAV, which is prevalent among humans. Accordingly, when a patient or subject is treated with viral-based gene therapy, there is a possibility that they will produce anti-viral neutralising antibodies (NAb) such as anti-AAV antibodies. Such NAb will neutralise the viral vector and thereby risk causing a significant reduction in therapeutic efficacy.

Several strategies are being developed to overcome the host immune response to AAV and extend treatment to more patients. One area of investigation is capsid modification, which includes the search for novel capsids that retain the efficient transduction of current serotypes (e.g. AAV2) while simultaneously presenting with low seroprevalence (e.g. AAV5). Other efforts have focused on postproduction modifications of AAV particles such as packaging into lipid-based nanoparticles (P. Guo, et al. 2019) or extracellular vesicles such as exosomes (György and Maguire 2018). More recently, preclinical data have raised the possibility of patient redosing after AAV co-administration with synthetic vaccine particles encapsulating rapamycin (SVP[Rapa], now ImmTOR) (Meliani, et al. 2018). As these technologies mature, it will be important to develop new ways to determine NAb status so that patients can be effectively treated with AAV gene therapy. Together, strategies to evade pre-existing NAb and prevention of their emergence after dosing promise to expand AAV gene therapy availability to an ever-greater number of patients.

Another avenue of investigation is the removal/depletion of NAb through antibody depletion techniques, such as depletion of NAb from patient plasma through apheresis/plasmapheresis. In plasmapheresis, blood removed from patients is separated into blood cells and plasma, the latter of which is discarded and replaced with an albumin solution. This approach can be used in the clinic to remove/deplete pathogenic immunoglobulins.

As a potential alternative to apheresis/plasmapheresis, targeted removal/depletion of immunoglobulin material from patient plasma is also known. As known from e.g. WO 2019/018439, an AAV binding antibody affinity matrix, attached to or immobilised on a substrate, may be used. As known from e.g. U.S. Pat. No. 10,286,087, an extracorporeal device for immunoadsorption, which includes a binding moiety which is specific for human IgG, may be used. Other conventional means may also be employed. Alternatives to external devices include the administration of enzymes (such as IgG cysteine proteases or IgG endoglycosidases) which digest human IgG. Methods of administering to the subject an agent which reduces Fc receptor binding of serum IgG molecules in the subject are disclosed inter alia in WO2016/012285, WO2020/016318 and WO2020/159970.

The transient nature of NAb reduction, whether via apheresis/plasmapheresis or otherwise, necessitates the use of quick and robust assays that enable monitoring of (AAV-) neutralising potential of patient NAb titres in near real time.

Assays do currently exist to screen for neutralising antibodies. One method to monitor NAbs in patient plasma is the in vitro transduction inhibition assay (TIA). TIA methods for assessing the presence of neutralising antibodies in a patient sample are known e.g. from Meliani et al. (Human Gene Therapy Methods, 26:45-53 (2015)). WO 2015/006743 describes such a transduction inhibition assay for detection of NAb titre to AAV, wherein recombinant AAV (rAAV) having a transgene encoding a reporter molecule is incubated with a sample from the patient. The mixture of virus and sample is subsequently incubated with target cells which can be infected with the rAAV. Following a 24-hour period for the AAV to transduce the reporter gene into the target cells, the expression of the reporter transgene is measured and compared with a control sample. The neutralising titre or NAb titre is defined as the dilution of the sample which results in 50% or greater inhibition of reporter gene by comparison with the control sample. The NAb titre values can be reported as a dilution range, e.g. 1:10 to 1:31; or can be reported as a “discrete titre” where a discrete titre of, say, 1:100 simply means that the NAb content is closer to 1:100 than it is to e.g. 1:200 or 1:50.

WO 2017/096162 describes a similar TIA method for detection of NAb titre to AAV and exemplifies a method that requires a 72-hour period for the AAV to transduce the reporter gene into the target cells before any signal is measured. Generally, known methods (as taught in e.g. Meliani et al., WO 2015/006743 or WO 2017/096162) involve a pre-assay step of plating out the target cells in advance (usually 24 hours prior to the assay or overnight) such that transduction is carried out on the immobilised/adhered cells.

Use of ELISA for quantification of antibody levels is known and is currently in use but, while much quicker to complete than the above-described transduction inhibition assay, it does not provide a functional readout of transduction inhibition since it only measures overall antibody levels (rather than NAb levels specifically) and—in contrast to the TIA method—does not provide a way to determine the NAb titre. Other methods, such FACS-based detection of cell-bound and labelled viral particles and qPCR detection of internalised vector genomes (P. Guo, et al. 2019), while expected to provide more accurate results of NAb potential than ELISA, do not inform on successful delivery of a functional vector genome in the cell nucleus.

Attempts to reduce the time taken for the TIA method tend to run into serious obstacles. Primarily there is the issue of assay sensitivity: by reducing the incubation time for the rAAV-reporter to transduce the target cells, there is a risk that insufficient signal will be produced. Accordingly, a patient who is reported to have low NAb titre may in fact have enough NAb in circulation to block transduction of the viral vector. Attempting to solve this problem by increasing the quantity of rAAV-reporter which is used leads to issues with creating a sufficiently robust assay signal (Z′-factor), in terms of distinguishing the resulting signal from the background. The Z-factor and Z′-factor are a widely-used parameter of assay quality in determining how well the signal is distinguished from the background and are discussed inter alia in Zhang et al. (Journal of Biomolecular Screening, Vol. 4, No. 2, 1999).

There is accordingly a need for assay methods which are capable of reliably measuring NAb titre without requiring a 24-hour incubation (or longer) of the rAAV-reporter with the target cells. It would be desirable to develop a robust assay to detect NAb titre levels which is capable of the rapid turnover of results within a day or alternatively overnight. There is a need for assay methods which are capable of reliably determining NAb titre even at low concentrations of antibody while still remaining robust (e.g. having a Z′ of greater than 0.5).

### SUMMARY OF THE INVENTION

The present invention relates to a luciferase-based transduction inhibition assay (TIA) with protocols that allow either same-day or next-day/overnight determination of a sample's inhibition titre (NAb titre). A crucial component of this method is a synthetic bright luciferase (“BrightLuc”), which can produce a robust luminescent signal at very low levels of expression. The present inventors have surprisingly found that the TIA provided herein is capable of detecting successful transduction after only 3 hours of transduction. In particular the present inventors have found that optimisation of various parameters (e.g. amount of vector used, cell numbers or MOI) can ensure a robust assay signal (Z′>0.5) even after only 3 hours of transduction.

Accordingly in one aspect the present invention provides a method for determining neutralising antibody (NAb) titre to a viral vector comprising a capsid of interest in a sample from a subject, the method comprising a transduction inhibition assay (TIA) using a luciferase which comprises the following steps:
(a) incubating particles of a viral vector comprising the capsid of interest in (1) one or more reference solutions comprising the sample at varying dilutions; and (2) at least one control solution; wherein the viral vector of part (a) comprises a recombinant vector genome comprising a transgene encoding the luciferase;
(b) exposing each of the solutions from step (a) to a population of target cells which are susceptible to infection by the viral vector of interest;
(c) waiting for a set interval of time to allow transduction to occur;
(d) adding a substrate for the luciferase to the reference and control solutions and measuring the signal (RLU) obtained from the luciferase;
(e) comparing the signal (RLU) obtained from the luciferase in the at least one control solution with the signal (RLU) obtained from the luciferase in the reference solutions; and
(f) calculating the NAb titre;
   wherein:
(i) the set interval of time in step (c) is less than 24 hours, optionally is 19 hours or less, optionally is 12 hours or less, optionally is 8 hours or less, optionally is 6 hours or less and optionally is 3 hours; and
(ii) the luciferase is a synthetic luciferase which provides enhanced luminescence relative to a firefly luciferase.

“NAb titre to a viral vector of interest” should be understood as referring to the NAb titre to the viral capsid. “Viral vector of interest” should be understood as referring to the viral vector comprising the capsid of interest.

A crucial component of this method is a synthetic bright luciferase (“BrightLuc”), which can produce a robust luminescent signal (Z′>0.5) at very low levels of expression. The present inventors have surprisingly found that the method of the invention provided herein is capable of detecting effective transduction, i.e. transduction resulting in gene expression, within only 3 hours of transduction.

Accordingly, a further aspect of the invention provides an AAV viral vector which comprises or encapsidates a recombinant vector genome comprising a transgene encoding a luciferase, wherein the luciferase is a synthetic luciferase which provides enhanced luminescence relative to a firefly luciferase.

The recombinant vector genome may be self-complementary. The recombinant vector genome may comprise a non-native promoter operably linked to the transgene encoding the luciferase. The promoter may be the CMV promoter. The AAV viral vector and/or the encoded luciferase may be as further defined herein.

The AAV viral vector may be provided together with a reagent which includes a substrate for the luciferase.

Accordingly, the present invention further provides a kit comprising an AAV viral vector of the invention which comprises or encapsidates a recombinant vector genome comprising a transgene encoding a luciferase, together with a reagent which includes a substrate for the luciferase. The kit may further comprise instructions for carrying out the method of the invention.

As set out in more detail below, a further advantage of the method of the invention is that it can be carried out on a suspension of target cells and does not require the target cells to be plated in advance. This permits target cells in suspension (including e.g. a suspension of thawed target cells) to be used directly in the method, without requiring a plating step in advance of the method.

Accordingly, the kit of the invention may further comprise a container comprising target cells. The target cells may be in suspension. The kit or the container may comprise insulating or cooling means.

It is preferred that the passage number of the target cells for use in the methods disclosed herein does not exceed 25.

Firefly luciferase is well known in the field. A reference firefly luciferase is provided here as SEQ ID NO: 1. Accordingly the synthetic luciferase may have enhanced luminescence relative to a firefly luciferase having a sequence according to SEQ ID NO: 1.

Synthetic luciferases are known. Such “synthetic” luciferases are generally derived from naturally-occurring luciferases but are modified—often significantly—to optimise one or more of their properties so as to provide e.g. enhanced luminescence, greater stability, smaller size, and so on. Such modifications generally involve reducing the size, e.g. by removing one or more of the protein subunits; and/or modifying the amino acid sequence of the luciferase. Such modifications may include conservative or non-conservative substitution, addition or deletion.

The enhanced luminescence of the synthetic luciferase may be determined by measuring the luminescence signal (RLU) of the synthetic luciferase and its substrate and the luminescence signal (RLU) of the firefly luciferase and its luciferine substrate under the same conditions, which allows a comparison to be made. The signal (RLU) of the synthetic luciferase and its substrate may be greater by at least 5-fold, at least 10-fold, at least 50-fold, at least 100-fold, at least 150-fold or more than the signal (RLU) of the firefly and its luciferine substrate. It is preferred that the synthetic bright luciferase for use with the method of the invention has a signal at least 80- to 100-fold or more than the above-defined firefly luciferase and its luciferine substrate.

Particularly preferred synthetic bright luciferases may be less than 50 kDa, less than 30 kDa, less than 25 kDa or less than 20 kDa. The synthetic bright luciferase may be ATP-independent.

The synthetic bright luciferase may use furimazine or coelenterazine as a substrate. The synthetic bright luciferase may use any known suitable derivatives or variants of furimazine or coelenterazine, or any other suitable known substrates. The choice of substrate will vary according to the synthetic bright luciferase chosen for the method and presents no difficulties to a person of skill in the relevant field.

The synthetic bright luciferase may comprise a sequence according to SEQ ID NO: 2 which has the commercial name “NanoLuc®” (Promega, U.S. Pat. No. 8,557,970). The synthetic bright luciferase may comprise a sequence having at least 90% or at least 95% identity with SEQ ID NO: 2. The synthetic bright luciferase may comprise a sequence which varies from SEQ ID NO: 2 by no more than one, no more than two, no more than three, no more than four or no more than five amino acids.

Alternatively, the synthetic bright luciferase may comprise a sequence according to SEQ ID NO: 3 which has the commercial name “TurboLuc®” (Thermo Scientific). The synthetic bright luciferase may comprise a sequence having at least 90% or at least 95% identity with SEQ ID NO: 3. The synthetic bright luciferase may comprise a sequence which varies from SEQ ID NO: 3 by no more than one, no more than two, no more than three, no more than four or no more than five amino acids.

Alternatively, the synthetic bright luciferase may comprise a sequence according to SEQ ID NO: 4 which has the commercial name “Lucia” (InvivoGen). The synthetic bright luciferase may comprise a sequence having at least 90% or at least 95% identity with SEQ ID NO: 4. The synthetic bright luciferase may comprise a sequence which varies from SEQ ID NO: 4 by no more than one, no more than two, no more than three, no more than four or no more than five amino acids.

Equivalents to the above synthetic bright luciferases are also encompassed within the scope of the method of the invention. Provided that the synthetic bright luciferase meets the test of having enhanced luminescence compared with a firefly luciferase as defined above, it is capable of being used in the method of the invention.

At least one control solution may comprise a negative control solution which lacks antibodies to the viral vector of interest. At least one control solution may comprise a first negative control solution which lacks antibodies to the viral vector of interest, and a second positive control solution which comprises a sufficient concentration of neutralising antibodies to maximally inhibit transduction of the viral vector of interest (as shown in Example 4 below, attaining complete 100% inhibition is not always practical or necessary). The positive control solution may be a solution of IVIG (in-vitro immunoglobulin). The IVIG solution will generally be at a sufficiently high concentration to ensure the maximal possible inhibition of the viral vector of interest. The IVIG may be at a concentration of at least 20 μg/ml, 30 μg/ml, 50 μg/ml or more. A concentration of at least 50 μdml is preferred.

Where at least one control solution comprises a positive control solution, the method may include a step of serially diluting the positive control solution and carrying out steps (a) to (d) above on the serial dilutions in order to establish the 50% inhibition level (EC50) of the positive control solution.

The sample from the patient will generally be a plasma sample.

The population of target cells may comprise at least 20,000 or 25,000 target cells. The population of target cells may comprise at least 50,000 target cells. The population of target cells may comprise at least 100,000 target cells, 150,000 target cells or more.

The target cells may be any mammalian cells which can be efficiently transduced by the viral vector being tested. In particular, the target cells may be HEK-293, HEK-293T, CHO, BHK, MDCK, 10T1/2, WEHI cells, COS, BSC 1, BSC 40, BMT 10, VERO, W138, MRCS, A549, HT1080, 293, B-50, 3T3, NIH3T3, HepG2, Saos-2, Huh7, HER, HEK, HEL, or HeLa cells. The target cells may be HEK293 cells, which may be HEK293T cells. The skilled person will readily be able to select a cell type for use with the method of the invention, based on the particular viral vector of interest being tested.

The viral particles referred to herein may be adeno-associated virus (AAV) viral particles. The AAV particles may have a capsid of or deriving from naturally-occurring AAV serotypes AAV1, AAV2, AAV3, AAV3B, AAV4, AAV5, AAV6, AAV7, AAV8, AAV9, AAV 10, AAV11, AAV 12, or a mixture thereof. AAV5 is of interest because it does not effectively transduce the cells conventionally used in a TIA and thus the method of the present invention provides a way to use TIA with AAV5 while still obtaining a robust signal. Accordingly in one aspect the AAV particles have a capsid of or deriving from AAV5.

Alternatively, the AAV particles may have a capsid which is non-naturally occurring/synthetic/engineered. Such capsids may be AAV3B-derived, and in particular may have a capsid comprising any one of SEQ ID NOs: 31, 46, 47, 54 or 56 from WO 2013/029030 (which correspond to SEQ ID NOs: 6, 7, 8, 9 or 10 of the present application, respectively), and in particular the capsid LKO3 (SEQ ID NO: 31 from WO 2013/029030 (SEQ ID NO: 6 of the present application)). The viral particles may have a capsid defined by SEQ ID NO: 5. The capsid may have at least 95%, 96%, 97%, 98% or 99% identity to the above capsids.

The viral particles may be lentiviral particles.

Step (a) of the method may comprise incubating the particles of the viral vector of interest at a concentration of between 1.7×10⁷ and 1.7×10⁵ vg/ml. An exemplified concentration is 8.3×10⁶ vg/ml.

The viral particles and target cells may be present at a ratio of vg:cells (multiplicity of infection (MOI)) which may be 250:1 or less, 200:1 or less, 100:1 or less, 50:1 or less, 25:1 or less, 10:1 or less, or 1:1 or less.

In particular the present inventors have found that optimisation of various parameters (e.g. amount of vector used, cell numbers or MOI) can ensure that the method of the invention gives rise to a robust assay signal (Z′>0.5) even after only 3 hours of transduction.

Accordingly, the method of the invention may have a calculated Z′ value of more than 0.5, 0.6 or more, 0.7 or more, or 0.75 or more.

The incubation step (a) may be for any length of time which allows any neutralising antibody present in the sample to bind to and neutralise the viral particles. This may be 1 hour, for 2 hours, for 3 hours, or more.

The sample diluent may comprise or may be healthy human plasma. Alternatively, it may comprise fetal bovine serum (FBS) which may be IgG-depleted FBS. The sample diluent may further comprise DMEM, which may be phenol-red free DMEM.

A dilution of the sample and/or the positive control may comprise a serial dilution. The serial dilution may comprise a 2-fold dilution factor. The serial dilution may comprise one or more of 1:2, 1:4, 1:8, 1:16, 1:32, 1:64, 1:128, 1:256, 1:1024, 1:2048, 1:4096 and so on; and/or may comprise 1 or more of 1:10, 1:50, 1:100, 1:200, 1:400, 1:800, 1:1600, 1:3200 and so on. The serial dilution may comprise between 5 and 15 dilutions. The serial dilution may comprise between 8 and 11 dilutions. The number of dilutions used is open to the skilled person.

In contrast with known methods, the method of the invention does not require the use of target cells which have been plated in advance. The method of the invention does not require the target cells to be immobilised or otherwise adhered to an assay plate. The present inventors have surprisingly found that a TIA method wherein the transduction is carried out on target cells which are in suspension is highly effective. Without being bound by any theory, the present inventors speculate that carrying out the transduction step on target cells in suspension may have a positive effect on the sensitivity of the assay.

Accordingly, step (b) and step (c) of the method of the invention may be carried out on a population of target cells in suspension. Step (b) may therefore be defined as a step of exposing each of the solutions from step (a) to a population of target cells in suspension, which target cells are susceptible to infection by the viral vector of interest.

Step (b) may include the step of providing the population of target cells on an assay plate and adding the solutions thereto. The assay plate may be a multi-well assay plate. An assay plate may be used irrespective of whether the target cells are in suspension.

An advantage of not requiring cells to be plated in advance of the assay is a reduction in complexity, as well as a reduction in time taken for the overall assay. Thus, the present invention provides an assay where the overall time taken, as well as the transduction time, is greatly reduced.

Accordingly, the method of the invention may therefore be carried out from start to finish within a period of 24 hours or less.

Step (d) may include a step of lysing the cells to release the synthetic luciferase into the solution, prior to adding the substrate. Alternatively, a substrate may be used which can penetrate the cells without the need for lysing them beforehand. As another alternative, the synthetic luciferase may be capable of being secreted from the target cells into the solution.

The NAb titre may be determined or quantified in a variety of ways.

The NAb titre may be calculated in step (f) above as the dilution of the reference solution at which the signal (RLU) obtained is 50% of the signal (RLU) obtained in the control solution. This may be calculated as a simple visual comparison between the dilutions of the reference solution(s) and the control solution(s).

The 50% inhibition may be compared relative to the negative control solution.

In step (f) of the method of the invention, the NAb titre may be calculated by using a nonlinear regression model to fit the reference solution data (i.e. the RLU values obtained from the reference solutions) to a curve and obtain a precise half-maximal value at which 50% neutralisation occurs. The non-linear regression model may be applied to the luminescent signal (RLU) from each sample dilution. The non-linear regression model may be applied to the luminescent signal (RLU) from each sample dilution after normalising with positive (maximal inhibition) and/or negative (0% inhibition) controls. NAb titre may be calculated by fitting a four-parameter variable-slope model to the luminescent signal from each sample dilution after normalising with both positive (maximal inhibition) and negative (0% inhibition) controls.

The NAb titre may be calculated as the interpolated titre at 50% transduction inhibition (T150).

The method of the invention is sensitive enough to distinguish between negative NAb samples and positive NAb samples with greater accuracy than ELISA. In particular, the method of the invention permits a population of patients to be rapidly and reliably stratified in a short period of time to determine their eligibility or suitability for gene therapy, based on their NAb titres to the viral vector which will be administered in the therapy.

Accordingly, another aspect of the invention provides a method of determining whether a patient is eligible for gene therapy using a viral vector comprising the capsid of interest, the method comprising determining the NAb titre (i.e. the titre of NAbs specific for the capsid of interest in the gene therapy viral vector) of the patient to said viral vector using the method defined above and comparing it with a determined or pre-determined threshold value wherein if the NAb titre is at or below the threshold value, the patient is eligible for gene therapy using the viral vector.

The threshold value at which a NAb titre is deemed to be acceptable, such that the patient is accordingly eligible for gene therapy, is something that a practitioner of skill in the art is able to determine without undue burden, for example based on clinical experience with vector particles possessing the same or similar particles.

Where a patient is defined as having a NAb titre that indicates that they are not currently eligible for gene therapy, one or more conventional methods known in the art may be employed to remove/deplete the NAb from their plasma, or at least to reduce the NAb titre to the threshold level or below, which will make the patient eligible for gene therapy. Techniques such as apheresis/plasmapheresis are clinical techniques which can be used in the clinic to remove/deplete pathogenic immunoglobulins. These techniques can reduce the NAb titres to AAV by 2-3-fold after each administration. However, several rounds of plasmapheresis may be required since some immunoglobulins have much longer half-life than others. Immunoglobulin G (IgG) levels in particular are characterised by a “rebound” phenomenon and can return to 40% of pre-apheresis levels within 48 h without concomitant immunosuppressive therapy.

The method of plasmapheresis referred to herein may be double filtration plasmapheresis (DFPP).

Accordingly another aspect of the invention provides a method of monitoring the progress of depletion of immunoglobulin which is specific for a viral vector comprising a capsid of interest, such as plasmapheresis or targeted depletion of immunoglobulin, in a patient wherein the method comprises the steps of:
(a) determining the patient NAb titre to a viral vector comprising a capsid of interest within a set interval of time following a round of immunoglobulin depletion by carrying out the method of the invention on a sample from the patient;
(b) determining whether the NAb titre following immunoglobulin depletion is below a predetermined threshold value for eligibility of gene therapy;
and optionally
(c) determining, based on the NAb titre following immunoglobulin depletion, whether a further round of immunoglobulin depletion is appropriate.

In a related aspect, the invention provides an AAV viral vector for use in a method of treating a genetic disorder, the method comprising:

(a) performing a method of immunoglobulin depletion on a patient;
(b) monitoring the progress of depletion of immunoglobulin using the method of the invention; and
(c) administering the AAV viral vector once the immunoglobulin is sufficiently depleted,
wherein the AAV viral vector comprises a transgene that encodes a polypeptide implicated in the genetic disorder, the AAV viral vector comprises a capsid, and the patient has antibodies to the capsid.

The set interval of time will be governed inter alio by the length of time required for successful transduction in the method of the invention. The set interval of time may be 24 hours or less, 19 hours or less, 12 hours or less, 9 hours or less or 6 hours or less.

The method of immunoglobulin depletion may be any such method which is known in the art, such as apheresis/plasmapheresis or targeted depletion of immunoglobulin. Targeted depletion of immunoglobulin may include methods relying on an affinity matrix or a binding moiety which is specific for immunoglobulin or specific for IgG or antibodies having specificity for the viral vector of interest.

The method of immunodepletion may specifically target immunoglobulin. The method of immunodepletion may use an extracorporeal device which binds IgG. Alternatively, the method of immunodepletion may comprise the administration of an enzyme (such as a IgG cysteine protease or IgG endoglycosidases) which digest human IgG. The method of immunodepletion may comprise a method of administering to the subject an agent which reduces Fc receptor binding of serum IgG molecules.

The agent or enzyme may be an IgG cysteine protease from a Streptococcus bacterium such as Streptococcus pyogenes, or an IgG endoglycosidase from a Streptococcus bacterium, such as Streptococcus pyogenes, Streptococcus equi or Streptococcus zooepidemicus, or from Corynebacterium pseudotuberculosis, Enterococcus faecalis, or Elizabethkingia meningoseptica. The agent or enzyme may have a sequence according to SEQ ID NO: 11 or SEQ ID NO: 12, or a fragment or variant thereof which has IgG cysteine protease activity.

This method of monitoring may be carried out with reference to an initial NAb titre which is obtained prior to any immunoglobulin depletion (for example, if the method of the invention is first used to determine whether the patient is eligible for gene therapy, their initial NAb titre will be known). “Initial NAb titre” is therefore to be understood as meaning the NAb titre of the patient pre-depletion and thus refers to pre-depletion levels of NAb in the patient. Accordingly, step (c) of the method of monitoring may include a step of comparing the NAb titre following immunoglobulin depletion with an initial NAb titre to determine whether the immunoglobulin depletion has had any effect.

The method of monitoring may be carried out following one or more further rounds of immunoglobulin depletion on the patient. Each additional round of immunoglobulin depletion may be performed on the same or consecutive days.

Thus, the method of monitoring may additionally include the following steps:
(d) determining the patient NAb titre to a viral vector comprising a capsid of interest within a set interval of time following a further round of immunoglobulin depletion by carrying out the method of the invention on a sample from the patient;
(e) determining whether the NAb titre following said further round of immunoglobulin depletion is below a predetermined threshold value for eligibility of gene therapy;
and optionally
(f) determining, based on the NAb titre following immunoglobulin depletion, whether a further round of immunoglobulin depletion is appropriate.

The step of determining whether yet a further round of immunoglobulin depletion is appropriate may include a step of comparing the NAb titre following the previous rounds of immunoglobulin depletion with one another, and optionally with the initial NAb titre, in order to determine whether the immunoglobulin depletion is having the intended effect.

It will be well within the capability of the person of skill in the art to determine whether a further round of immunoglobulin depletion is appropriate. If for example there is very little difference between the initial NAb titre and the NAb titres following more than one round of immunoglobulin depletion, it might be deemed inappropriate to subject the patient to yet a further round of immunoglobulin depletion. Alternatively, if the NAb titre following more than one round of immunoglobulin depletion is very close to the determined or pre-determined threshold value which would render the patient eligible for gene therapy, it may be deemed appropriate to subject the patient to yet a further round of immunoglobulin depletion. Generally, if NAb levels are still above threshold, another round of immunoglobulin depletion is appropriate, unless the previous round has not had a significant effect.

Thus, the method of monitoring may additionally include the following steps:
(g) determining the NAb levels in the patient within a set interval of time following the previous round of immunoglobulin depletion by carrying out the method of the invention on a sample from the patient;
(h) determining whether the NAb titre following the previous round of immunoglobulin depletion is below a determined or pre-determined threshold value for eligibility of gene therapy;
and optionally
(i) determining whether a further round of immunoglobulin depletion is appropriate.

It will be understood that immunoglobulin levels rebound following depletion, such that it is generally desirable to carry out the method of monitoring, and all required rounds of immunoglobulin depletion, with a period of no more than four days, preferably within three days, and most preferably within 48 hours or less. Accordingly all of steps (a) to (c), (a) to (f) or (a) to (i) may be carried out within a period of 4 days or less, within 72 hours or less, within 48 hours or less or within 24 hours.

It is preferred that the further round(s) of immunoglobulin depletion of step (c), step (f) and step (i) is/are carried out within 48 hours or less of the previous round of immunoglobulin depletion, and preferably the day after the previous round of immunoglobulin depletion.

Each further round of immunoglobulin depletion may be carried out within 24 hours of the previous round of immunoglobulin depletion.

As demonstrated in the Examples below, “consecutive” cycles of plasmapheresis (meaning that each cycle is delivered the day after the previous cycle) are capable of reducing the patient's initial NAb titre to a level that is appropriate for clinical trial eligibility and/or for gene therapy. In particular, five cycles of plasmapheresis over five consecutive days are predicted to reduce a patient's initial NAb titre by up to 94%.

Accordingly the method may comprise:
(a) two rounds of immunoglobulin depletion over the course of two days;
(b) three rounds of immunoglobulin depletion over the course of three days;
(c) four rounds of immunoglobulin depletion over the course of four days; or
(d) five rounds of immunoglobulin depletion over the course of five days.

Accordingly it will be well understood that the method of the invention, which permits an accurate measurement of NAb titre to be obtained in less than 24 hours, provides a clear advantage over currently known methods which require incubation/transduction periods of 24 hours or more before the result can be obtained.

### DETAILED DESCRIPTION

### General Definitions

Unless defined otherwise, technical and scientific terms used herein have the same meaning as commonly understood by a person skilled in the art to which this invention belongs.

In general, the term “comprising” is intended to mean including but not limited to.

In some embodiments of the invention, the word “comprising” is replaced with the phrase “consisting of” or the phrase “consisting essentially of”. The term “consisting of” is intended to be limiting.

As used herein, “gene therapy” is the insertion of nucleic acid sequences (e.g., genes) into an individual's cells and/or tissues to treat a disease, such as hereditary diseases where a defective mutant allele is replaced or supplemented with a functional one. Acquired diseases such as blood clotting disorders can be treated by gene therapy.

“Adeno-associated viruses” (AAV), from the parvovirus family, are small viruses with a genome of single stranded DNA. These viruses are useful as gene therapy vectors. Because AAV are not associated with pathogenic disease in humans, AAV vectors are able to deliver therapeutic proteins and agents to human patients without causing substantial AAV pathogenesis.

“Lentiviruses” are a genus of retrovirus which can integrate a significant amount of viral cDNA into the DNA of the host cell and can efficiently infect nondividing cells, making them an efficient means of gene delivery. Lentiviruses can also be used to stably over-express certain genes, thus allowing researchers to examine the effect of increased gene expression in a model system. Another common application for lentiviral vectors is to introduce a new gene into human or animal cells. For example, a genetic disorder such as hemophilia may be corrected in this manner.

An “AAV vector” or simply “vector” is derived from the wild type AAV by using molecular methods to remove the wild type AAV genome, and replacing it with a non-native nucleic acid, such as a therapeutic gene expression cassette. Typically, the inverted terminal repeats of the wild type AAV genome are retained in the AAV vector. An AAV vector is distinguished from an AAV, since all or a part of the viral genome has been replaced with a transgene cassette, which is a non-native nucleic acid with respect to the AAV nucleic acid sequence.

The term “bind,” “binding,” or “react with” means that the antibody, virus capsid or capsid protein interacts at the molecular level. Thus, a capsid protein that binds to or reacts with an antibody interacts with the antibody at the molecular level.

A “neutralising antibody” or “NAb” is an antibody which binds to a viral vector in such a manner as to prevent that viral vector from transducing a target cell.

It is known that capsid proteins of different serotypes can be cross-reactive with antibodies against a particular serotype. Thus, for example an antibody such as a NAb against a given AAV serotype, e.g. AAV2, may also bind to one or more other AAV serotypes. A “serotype” is traditionally defined on the basis of a lack of cross-reactivity between antibodies to one virus as compared to another virus. Such cross-reactivity differences are usually due to differences in capsid protein sequences/antigenic determinants (e.g., due to VP1, VP2, and/or VP3 sequence differences of AAV serotypes).

AAV include various naturally and non-naturally occurring serotypes. Such non-limiting serotypes include AAV-1, -2, -3, -3B, -4, -5, -6, -7, -8, -9, -10, -11, -rh74, -rhlO and AAV-2i8.

Viral (e.g., AAV or lentivirus) vectors can be used to introduce/deliver polynucleotides stably or transiently into cells. The term “transgene” is used to conveniently refer to such a heterologous polynucleotide that can be introduced into a cell or organism by way of a viral vector. Transgenes broadly include any polynucleotide, such as a gene that encodes a polypeptide or protein, a polynucleotide that is transcribed into an inhibitory polynucleotide (e.g., siRNA. miRNA, shRNA), or a polynucleotide that is not transcribed (e.g., lacks an expression control element, such as a promoter that drives transcription).

As used herein, the term “recombinant,” as a modifier of viral vector, such as recombinant AAV vectors or recombinant lentiviral vectors, as well as a modifier of sequences such as recombinant polynucleotides and polypeptides, means that the compositions have been manipulated (i.e. engineered) in a fashion that generally does not occur in nature. A particular example of a recombinant AAV would be where a polynucleotide that is not normally present in the wild-type AAV is within the AAV particle and/or genome. A particular example of a recombinant polynucleotide would be where a transgene encoding a protein is cloned into a vector. Although the term “recombinant” is not always used herein in reference to AAV vectors, as well as sequences such as polynucleotides and polypeptides, recombinant forms of AAV and AAV vectors, and sequences including polynucleotides and polypeptides, are expressly included in spite of any such omission.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

For the purpose of this invention, in order to determine the percent identity of two sequences (such as two polypeptide sequences), the sequences are aligned for optimal comparison purposes (e.g., gaps can be introduced in a first sequence for optimal alignment with a second sequence). The amino acids at each position are then compared. When a position in the first sequence is occupied by the same amino acid as the corresponding position in the second sequence, then the sequences are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 1, then in that instance SEQ ID NO: 1 would be the reference sequence. To assess whether a nucleotide sequence is at least 80% identical to SEQ ID NO: 1 (an example of a reference sequence), the skilled person would carry out an alignment over the length of SEQ ID NO: 1, and identify how many positions in the test sequence were identical to those of SEQ ID NO: 1. If at least 80% of the positions are identical, the test sequence is at least 80% identical to SEQ ID NO: 1. If the sequence is shorter than SEQ ID NO: 1, the gaps or missing positions should be considered to be non-identical positions.

For the avoidance of doubt, it will be understood that references to “at least 80% identity,” “at least 90% identity,” “at least 95% identity” and/or “at least 98% identity” should all be read as implicitly including 100% identity.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

The term “immune response” is meant to refer to any response to an antigen or antigenic determinant by the immune system of a vertebrate subject. Exemplary immune responses include humoral immune responses (e.g. production of antigen-specific antibodies) and cell-mediated immune responses (e.g. lymphocyte activation or proliferation).

The term “neutralising antibody titre” or “NAb titre” as used herein refers to the extent of neutralising antibody (NAb) present in a sample such as a sample from a patient or subject. For the purposes of this application, antibody titre is expressed as a dilution of the sample at which 50% inhibition of transduction is observed. For example, if a test for anti-AAV NAb shows that a dilution of 1:10 results in a 50% inhibition of transduction of the AAV vector, then the anti-AAV NAb amount (titre) will be 1:10.

The “Z-factor” is a reference to the robustness of an assay and is a reference to the screening window coefficient (Zhang et al.). It has been widely adopted as a useful tool for comparison and evaluation of the quality of assays. In particular the calculation of the Z-factor replaces the earlier comparisons of signal-to-noise ratio (defined as the difference between the mean signal value and mean background values divided by the standard deviation of the background) and the signal-to-background ratio (calculated as the mean signal values over the mean background values). The Z-factor can be calculated as:

$$Z = \frac{\vert {μ}_{s} - {μ}_{c} \vert - ( 3 {σ}_{s} + 3 {σ}_{c} )}{\vert {μ}_{s} - {μ}_{c} \vert}$$

Alternatively, it can be defined as

$$Z = 1 - \frac{( 3 {σ}_{s} + 3 {σ}_{c} )}{\vert {μ}_{s} - {μ}_{c} \vert}$$

Where μₛ is the mean of the sample; μₖ is the mean of the controls; σₛ is the standard deviation of the samples; and σ_{c} is the standard deviation of the controls.

The “Z′-factor” is a characteristic parameter for the quality of an assay by reference to its control data alone. A Z′ value which is negative or close to zero indicates that the assay is not suitable for generating useful data. A Z′ value of >0.5 is an indicator that the assay is suitably robust. Z′-factor is therefore appropriate for calculating overall assay quality. It is defined as:

$${Z}^{′} = 1 - \frac{( 3 {σ}_{c +} + 3 {σ}_{c -} )}{\vert {\underline{μ}}_{c +} - {\underline{μ}}_{c -} \vert}$$

Where μ_{c+} is the mean of the positive control; μ_{c+} is the mean of the negative control; a, is the standard deviation for the positive control; and σ_{c-} is the standard deviation for the negative control.

In view of the data provided in the Examples below as well as the statements of invention set out above, it will be readily understood that the method of the present invention is more accurate than corresponding ELISA methods in that it minimises false positives and false negatives (FIG. 13). Another advantage of the present invention lies in the provision of a rapid TIA which permits a determination of NAb titre after each round of immunoglobulin depletion (e.g. via plasmapheresis) before ‘rebound’ of the immunoglobulins can occur. This in turn permits a determination to be made of whether immunoglobulin depletion has been successful enough to render a patient suitable for gene therapy (i.e. by depleting their NAb to the viral vector which will be used in gene therapy) and/or to determine whether one or more further rounds of immunoglobulin depletion would be appropriate.

The method of the invention is sensitive enough to distinguish between negative NAb samples and positive NAb samples with greater accuracy than ELISA. In particular, the method of the invention permits a population of patients to be rapidly and reliably stratified in a short period of time to determine their eligibility or suitability for gene therapy, based on their NAb titres to the viral vector which will be administered in the therapy.

Accordingly it will be well understood that the method of the invention, which permits an accurate measurement of NAb titre to be obtained within 6 hours or less, provides a clear advantage over currently known methods which require incubation/transduction periods of 24 hours or more before the result can be obtained.

### DESCRIPTION OF THE FIGURES

The present invention will now be described by way of non-limitative example with reference to the following figures, in which:

FIG. 1 shows a reaction diagram for a particularly preferred BrightLuc of interest (commercial name NanoLuc®) showing that the conversion of its substrate and the release of light is ATP-independent.

FIG. 2 (2A) The FLGO97 plasmid was used to generate the AAV-BrightLuc (AAV-BL) reporter, designated RC-01-31, also referred to herein as scAAV-NLuc. (2B) Titre of the AAV-BrightLuc reporter used in the examples together with the titre of the AAV-eGFP vector used as a comparator. Titres were determined by qPCR and capsid ELISA.

FIG. 3 shows the results of measuring the luminescent signal produced by AAV-BrightLuc reporter vector. Panels A-D show the performance of the luminescence assay signal over 1 hour after application of Nano-Glo reagent form 1×10⁵ HEK293T cells/well incubated over 6 hours with the indicated dilutions of AAV-BrightLuc reporter vector. Panels E-H show the results after 24 hours' incubation. (3A) The raw luminance signal after 6 hours transduction. (3B) The S:B (signal-to-background) calculated against untransduced (NT) cells at 6 hours. (3C) The coefficient of variation (“CV”) values (defined throughout as (standard deviation/average)*100) following 6 hours of incubation. (3D) The Z′ values following 6 hours of incubation. (3E) The raw luminance signal after 24 hours of transduction. (3F) The S:B (signal-to-background) calculated against untransduced (NT) cells at 24 hours. (3G) The CV values following 24 hours of incubation. (3H) The Z′ values following 24 hours of incubation. (3I) Summary of the assay conditions tested.

FIG. 4 shows the results of measuring the luminescent signal from 1/300,000, 1/30,000 and 1/3,000 dilutions of the AAV-BrightLuc reporter vector, at 3, 6 or 24 hours of transduction of 2.5×10⁴, 5×10⁴ or 10×10⁴ cells/well. The timepoints tested were 3 hours (4A-4D), 6 hours (4E-4H) and 24 hours (4I-4L). Signal from untransduced cells is shown in (4A), (4E) and (4I). Signal from 1/300,000 vector dilution is shown in (4B), (4F) and (4J). Signal from 1/30,000 vector dilution is shown in (4C), (4G) and (4K), while signal from 1/3,000 dilution of vector is shown in (4D), (4H) and (4L). (4M): Luminescent signal performance after 3 h, 6 h or overnight transduction with of AAV-BL at indicated MOIs. Panels (i) and (ii) represent the signal obtained at all tested MOIs and timepoints for 25,000 and 50,000 cells respectively.

FIG. 5 shows the assay sensitivity using IVIG. (5A) Testing of inhibition with 1:2000 IVIG after 1 hour incubation in the presence of 1% negative plasma (i.e. 1% negative plasma with normal growth media (DMEM) as the diluent), using the indicated dilutions of AAV-BrightLuc reporter vector over 6 hours of transduction. (5B) percentage of luminescent signal remaining after inhibition of the indicated dilutions of scAAV-Nluc after 6 hours of transduction.

FIG. 6 shows the results of a determination of the neutralising titre of IVIG. 1/1,500 (6A) and 1/15,000 (6B) dilutions of AAV-BrightLuc reporter vector were neutralised over 1 hour with a series of IVIG dilutions in the presence of 2% negative plasma. 1×10⁵ cells/well were then incubated with the neutralised material for 6 hours. Untransduced (NT) and cells transduced in the presence of 2% negative plasma were used as positive and negative controls respectively. EC50 values are listed.

FIG. 7 shows a comparison between the 6-hour TIA protocol of the invention using an AAV-BrightLuc reporter vector (“rTIA”); and a more conventional TIA using an AAV-eGFP reporter vector (“cTIA”) or an AAV-Firefly luciferase reporter vector. (7A) The correlation between TI50 values generated using the “rTIA” and “cTIA” protocols, using 26 samples. (7B) The correlation between discrete titre values generated using the “cTIA” and “rTIA” protocols, using 36 samples. (7C) Comparison of luminescent signal obtained from AAV-BrightLuc and AAV-Firefly vectors after 6 h of transduction.

FIG. 8 shows a comparison between the results of the TIA protocol of the invention using an AAV-BrightLuc reporter vector after 6 hours and 16 hours.

FIG. 9 shows a comparison between the TIA protocol of the invention using an AAV-BrightLuc reporter vector (“rTIA”) after 16 hours; and a more conventional TIA using an AAV-eGFP reporter vector (“cTIA”). (9A) The correlation between TI50 values generated using the “rTIA” and “cTIA” protocols, using 25 samples. (9B) The correlation between discrete titre values generated using the “cTIA” and “rTIA” protocols, using 33 samples. The sample diluent was human plasma sample “TCS137”.

FIG. 10 shows the TI50 values which were obtained by the TIA protocol of the invention using an AAV-BrightLuc reporter vector after 16 hours in various sample diluents and concentrations. “TCS137”=negative patient plasma. “IgGD”=IgG-depleted FBS.

FIG. 11 shows a comparison between the TIA protocol of the invention using an AAV-BrightLuc reporter vector (“rTIA”) after 16 hours; and a more conventional TIA using an AAV-eGFP reporter vector (“cTIA”). (11A) The correlation between T150 values generated using the “rTIA” and “cTIA” protocols, using 24 samples. (11B) The correlation between discrete titre values generated using the “cTIA” and “rTIA” protocols, using 32 samples. The sample diluent was phenol red-free DMEM supplemented with 50% IgG-depleted FBS.

FIG. 12 shows a comparison of T150 values obtained using the AAV-BrightLuc reporter vector after 6 hours and after 16 hours. (12A) Correlation between T150 values obtained with the 16-hour AAV-BrightLuc reporter vector using either negative patient plasma (TCS137, x-axis) or IgG-depleted FBS (y-axis) as the sample diluent, using 32 samples. (12B) Correlation between TI50 values obtained with either the 6-hour AAV-BrightLuc reporter vector (x-axis) or the 16-hour AAV-BrightLuc reporter vector (y-axis) using negative patient plasma (“TCS137”) as the sample diluent. (12C) Correlation between TI50 values obtained with either the 6-hour AAV-BrightLuc reporter vector (x-axis) or the 16-hour AAV-BrightLuc reporter vector (y-axis) using IgG-depleted FBS as the sample diluent, using 28 samples. (12D) Summary of the results from (12A), (12B) and (12C).

FIG. 13 shows a comparison of TIA and ELISA triaging in haemophilia B patients. The overnight TIA protocol was used to determine TI50s, while an AAV ELISA was used to determine the anti-AAV antibody titre for 62 patient samples. A TI50 of 1:8 and anti-AAV antibody titre of 10 μg/mL were used as cut-offs to determine patient eligibility for gene therapy. Open circles (bottom left) indicate samples found to be negative (i.e. having a neutralising titre that is acceptably low enough to allow AAV gene therapy) by the ELISA and the TIA methods. Open squares (bottom right) indicate samples found to be negative by the TIA method but not by the ELISA method, i.e. false positives. Open crossed circles (top left) indicate samples found to be negative by the ELISA method but not by the TIA method, i.e. false negatives. Solid circles (top right) indicate samples found to be positive (i.e. having a neutralising titre that is higher than the designated cut-off point). Dashed circles indicate where the values obtained via ELISA represent a false positive/false negative once the correct values have been ascertained using the method of the invention.

FIG. 14 shows an analysis of samples from 36 patients undergoing double-filtration plasmapheresis (DFPP) and compares an anti-AAV ELISA with a GFP-based TIA. (14A) Analysis of samples before (“pre”) and after (“post”) each DFPP round using an anti-AAV ELISA assay with an IVIG standard curve (mean+SEM). (14B) AAV neutralising titre analysis of pre- and post-DFPP samples using a GFP-based TIA. Horizontal lines show group averages. (C) Comparison of ELISA and GFP-based TIA triaging using cut-offs of 10 μg/mL and 1:100 respectively. Open circles (bottom left) indicate samples found to be negative (i.e. having a neutralising titre that is acceptably low enough to allow AAV gene therapy) by the ELISA and the TIA methods. Open squares (bottom right) indicate samples found to be negative by the TIA method but not by the ELISA method, i.e. false positives. Open dashed circles (top left) indicate samples found to be negative by the ELISA method but not by the TIA method, i.e. false negatives. Solid circles (top right) indicate samples found to be positive (i.e. having a neutralising titre that is higher than the designated cut-off point).

FIG. 15 shows an analysis of samples with or without “consecutive” cycles of DFPP using the TIA of the invention (BL-TIA). (15A) Analysis of samples before (“pre”) and after (“post”) each DFPP round using BL-TIA; solid circles indicate “consecutive” cycles and open circles indicate “non-consecutive.” (15B) T150 reduction and regression analysis after 3 “consecutive” DFPP cycles (n=4). (C) Comparison of T150 reduction between 5 “non-consecutive” and either 3, 4 or 5 “consecutive” DFPP cycles. Note that for cycle 5 “Pre”, the topmost solid circle (solid line) overlaps with an open circle (dashed line) so that only the solid circle can be seen.

### EXAMPLES

Two protocols were developed that can be completed within <9 hours or overnight, respectively referred to as the 6 and 16 hour protocols. Both protocols use the same amount of scAAV-Nluc reporter vector (i.e. 1/6,000, equivalent to 8.3×10⁶ vg/ml) and produce equivalent T150 values over 30 samples tested.

Assay sensitivity and robustness were considered in parallel during development. On one hand, sensitivity of these methods to the neutralising activity of a sample is inversely proportional to the amount of vector used, since the higher the amount of vector used during inhibition, the less likely it is for the assay signal to be meaningfully inhibited via vector neutralisation. On the other hand, the robustness of the assay signal is directly proportional to the amount of vector used because successful transduction and transgene expression depend on MOI. Therefore, to balance sensitivity and robustness for the assays, the smallest amount of vector was sought that would still yield a robust assay signal (Z′>0.5) within a short time frame. Any new batch of reporter scAAV-Nluc vector is recommended to be serially diluted to empirically determine the smallest dilution that still yields a robust assay signal, regardless of the estimated vg/ml or vp/ml titre.

The number of cells/well, the amount of vector used, and the time of incubation were all optimised. Within 3 h, a 1/3000 dilution of the AAV-Nluc vector (vg:cell MOI of 8.33:1) results in a marginal assay signal (Z′ value between 0 and 0.5). Within 6 h, a robust signal is obtained with a MOI of 0.83:1, simultaneously satisfying the requirements for sensitivity, robustness and the quick turnover of results. These conditions translate to a concentration of 8.3×10⁶ vg/ml during vector neutralisation for 1 hour at 37° C. and were not altered when extending the within-day protocol with a 16 hour transduction step.

### Materials and Methods

### Samples

Several plasma samples were used during the development and testing of the within-day and over-night protocols. Where indicated, test samples were diluted using a healthy human plasma sample (“TCS137”) with low anti-AAV neutralising activity. For a list of all other tested samples, sourced from TCS Biosciences and LGC, see below:

| | cTIA discrete |
|---|---|
| sample ID | titre |
| LGC 3 | 1/80 |
| LGC 30 | 1/80 |
| LGC 33 | 1/80 |
| LGC 34 | 1/80 |
| TCS 67 | 1/100 |
| TCS 79 | 1/100 |
| TCS 80 | 1/100 |
| TCS 81 | 1/100 |
| TCS 105 | 1/100 |
| TCS 118 | 1/100 |
| LGC 35 | 1/100 |
| LGC 47 | 1/100 |
| LGC 49 | 1/100 |
| LGC 50 | 1/100 |
| TCS 27 | 1/200 |
| TCS 55 | 1/200 |
| TCS 70 | 1/200 |
| TCS 85 | 1/200 |
| TCS 106 | 1/200 |
| LGC 9 | 1/200 |
| TCS126 | 1/200 |
| TCS127 | 1/200 |
| TCS 73 | 1/400 |
| TCS 92 | 1/400 |
| TCS 93 | 1/400 |
| TCS 117 | 1/400 |
| TCS 40 | 1/800 |
| TCS 44 | 1/800 |
| TCS 56 | 1/800 |
| TCS 59 | 1/800 |
| TCS 62 | 1/800 |
| TCS 64 | 1/800 |
| TCS 87 | 1/800 |
| TCS 108 | 1/800 |
| TCS 112 | 1/800 |
| TCS 113 | 1/800 |
| TCS 89 | 1/1600 |
| TCS 94 | 1/1600 |
| TCS 95 | 1/1600 |
| TCS 99 | 1/1600 |
| TCS 100 | 1/1600 |
| TCS 97 | 1/3200 |
| TCS 98 | 1/3200 |
| TCS 111 | 1/6400 |
| TCS 101 | 1/12800 |

### Assay Protocol

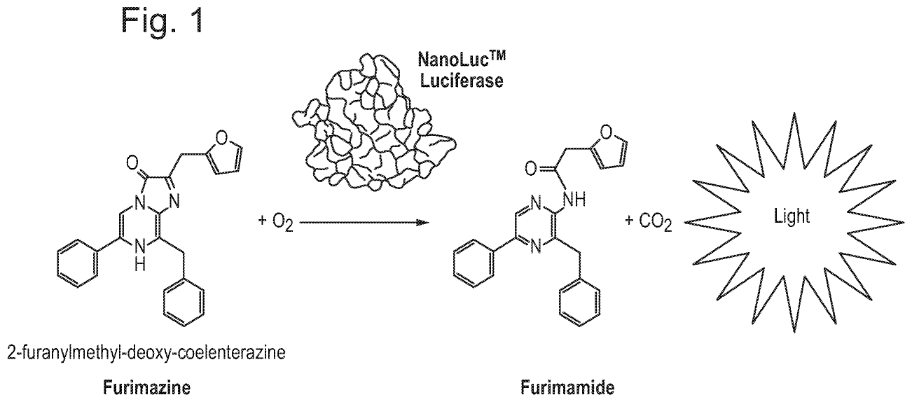

An in vitro transduction inhibition assay capable of delivering a result in 6 hours or 16 hours (i.e. overnight) was developed and tested. Upon transduction, the AAV-BrightLuc reporter vector (termed “scAAV-Nluc” using the capsid according to SEQ ID NO: 5) delivers a self-complementary AAV genome that expresses the luciferase known commercially as NanoLuc® from the upstream CMV promoter. The NanoLuc® luciferase is a small (19.1 kDa) ATP-independent luciferase that efficiently converts furimazine to furimamide (FIG. 1) to generate bright and long lasting (glow-type) luminescence. The present examples are set up to detect NAbs for the AAV-capsid used herein, but it will be understood that the principle of the assay design is generally applicable.

In this assay, the positive control consists of sample diluent spiked with intravenous immunoglobulins (IVIG) at 1:1000 dilution (equivalent to 50 micrograms/milliliter), while the negative control is sample diluent alone. Both controls are supplemented with scAAV-Nluc vector, and the fold difference in signal (negative/positive control) is expected to exceed 100-fold. The sample diluent can be either human plasma (sourced as set out above) or a dilution of commercially available IgG-depleted FBS (see Example 9) in phenol-red free DMEM (Thermo Scientific cat. no. 31053-028), lacking in significant AAV neutralising activity.

The assay is carried out by using two 96-well plates: a v-bottom plate (Greiner Cellstar product no. 651180), in which a test sample is serially diluted and mixed with scAAV-Nluc over 1 hour, is termed the “neutralisation plate”. A white, sterile, TC (tissue culture) treated plate (Corning 3917) in which the neutralised sample and reporter vector mixtures are incubated with 1×10⁵ or 1.5×10⁵ HEK239T cells/well for 16 hours or 6 hours respectively is termed the “assay plate”.

Typically, 66 μl of sample is used to generate a serial dilution by moving and mixing 33 μl of this sample with 33 μl of pre-dispensed sample diluent. Each resulting sample dilution is then supplemented with 66 μl of diluted scAAV-Nluc vector such that the final reporter concentration is 8.3×10⁶ vg/ml. After 1 hour incubation at 37° C., the sample and vector mixtures are transferred to the assay plate by mixing in quadruplicate 15 μl of sample and vector mix to a 60 μl volume containing 150,000 HEK293T cells in suspension (6 hour protocol) or in triplicate 25 μl of sample and vector mix to a 50 μl volume containing 100,000 cells in suspension (16 hour protocol). By carrying out the transduction on cells in suspension, the method of the invention avoids the need for preparing the cells on plates in advance, in contrast with conventional reporter-based TIA methods which require the cells to be plated well in advance. Timings may differ according to the precise protocols employed, but in conventional reporter-based TIA methods, cells are generally plated at least overnight or 24 hours in advance of the assay.

After incubation (for 6 hours or 16 hours) under normal growth conditions (37° C., 5% CO2), each well of the assay plate is supplemented with 75 μl of Nano-Glo assay reagent containing the substrate for the luciferase (Promega N1110) prepared according to manufacturer's instructions. Luminescence is read on a plate reader using a reading height of 5 mm above the plate, and integration time of 0.5 ms.

### Analysis

In this assay, the neutralising titre of a plasma or serum sample is calculated by fitting a four-parameter variable-slope model to the luminescent signal from each sample dilution after normalising with both positive (100% inhibition) and negative (0% inhibition) controls. The primary numerical output is the interpolated titre at 50% transduction inhibition, which the present inventors have termed “T150.” T150 ranks samples in proportion with their NAb content. An optional readout, which is employed in a conventional FACS-based transduction inhibition assay, is the discrete neutralising titre. The discrete neutralising titre is defined as the highest sample dilution in which the level of luminescence is ≥50% of the level of the assay positive control.

To compare results between two methods (e.g. cTIA vs 6 hour rTIA), a Spearman rank correlation value (r) is computed for the resulting T150s or discrete neutralising titres.

### Example 1—Design of the scAAV Reporter Cassette: CMV-NanoLuc®-SV40 pA

The luciferase NanoLuc® (Hall, et al. 2012) was chosen for inclusion in an AAV reporter vector, using a CMV promoter which can drive robust gene expression in multiple cell lines. To boost expression rates, a self-complementary vector genome which bypasses the need for second strand synthesis was used. Together, these features form the basis of the scAAV-Nluc (also referred to herein as “AAV-BrightLuc” and “RC-01-31”) reporter vector.

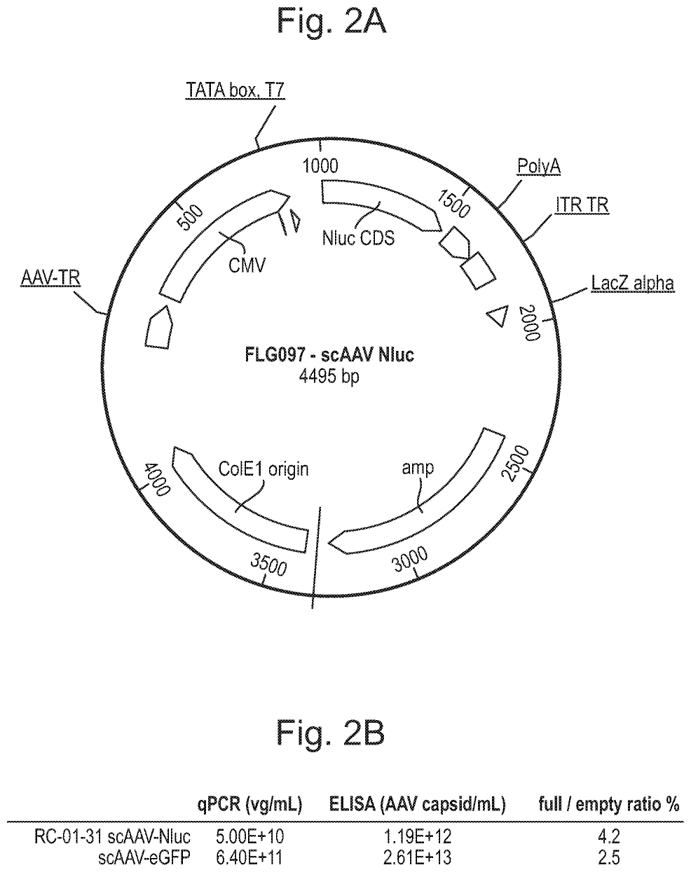

To provide the most accurate measure of the vg/ml titre of the RC-01-31 vector, qPCR and capsid ELISA were carried out in parallel with a separate reporter vector, which is otherwise identical to the scAAV-Nluc apart from the inclusion of a transgene encoding GFP (green fluorescent protein) instead of a luciferase. This scAAV-eGFP (also referred to herein as (“AAV-eGFP”) reporter vector is currently used in a current FACS-based transduction inhibition assay (also referred to herein as “cTIA” to distinguish it from the rapid TIA (“rTIA”) of the invention). qPCR revealed a ^{˜}10× difference between these vectors, which was consistent with the results obtained using capsid ELISA (FIG. 2B).

### Example 2—Evaluation of Luciferase Signal after Transduction with AAV-BrightLuc Over 6 Hours and 24 Hours

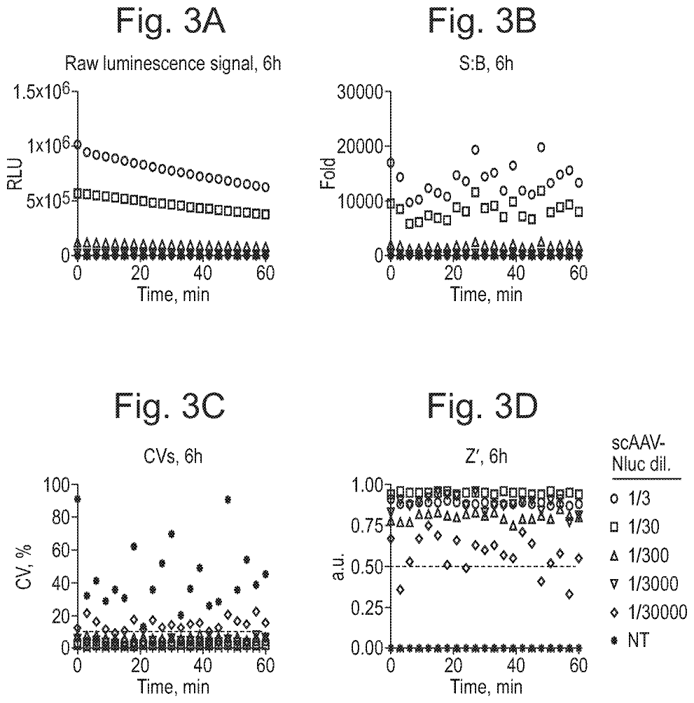

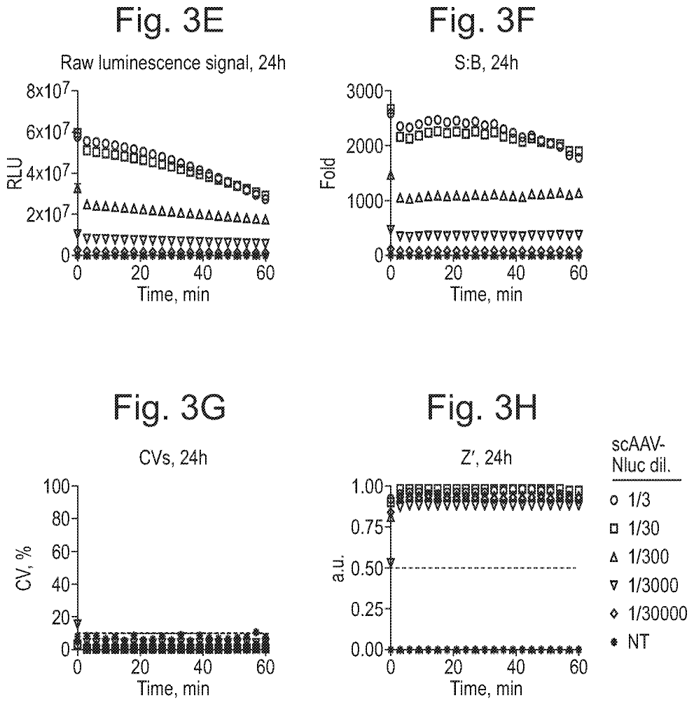

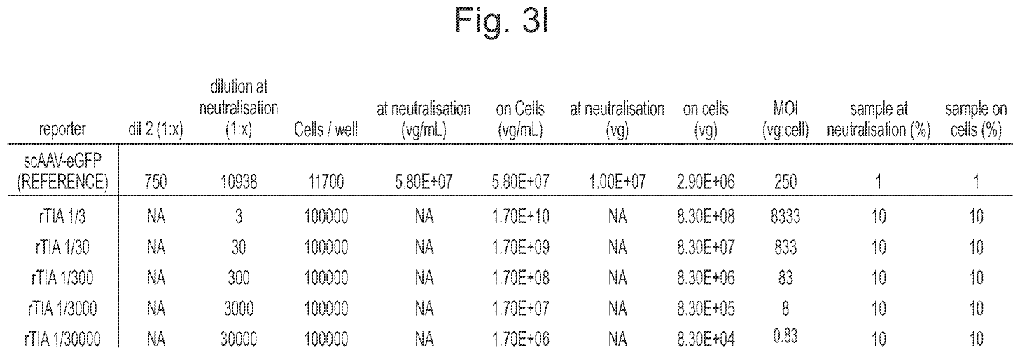

A conventional FACS-based transduction inhibition assay (cTIA) was used as a starting point in the development of the present method. Use of the scAAV-eGFP vector stock in the cTIA protocol includes application of 2.9×10⁶ vg to about 1.2×10⁴ HEK293T cells (per well) yielding a multiplicity of infection (MOI, defined as the ratio between the number of vector genomes and the number of host cells) of 250 (vg:cell) in a 50 μl volume (FIG. 3I).

To determine whether the AAV-BrightLuc vector could provide a luminescent signal within 6 hours of infection, several dilutions of this vector were applied to a suspension of HEK239T cells in a 50 μl volume. To provide as many available cell-association sites as possible for AAV, 1×10⁵ HEK239T cells (roughly 8× more than cTIA) were used during incubation. In addition to improving transduction rates, this number of cells allowed probing of a range of MOIs (8333:1-0.83:1, FIG. 3I).

Phenol red can hinder detection of weak luminescent signals and therefore phenol red-free DMEM (Catalogue no. 21063-029) was used to ensure efficient detection. 10% negative (“10%-ve”) plasma diluted in DMEM was also included during incubation, because it is equivalent to the maximum amount of FBS (foetal bovine serum) supplement used to routinely maintain HEK293T cells. After 6 hours or 24 hours of transduction, 50 μl of Nano-Glo reagent was added to each well and the assay plate was read every 3 minutes over 1 hour using a Molecular Devices i3× spectrophotometer. The resulting raw values were plotted against time for both 6-hour and 24-hour timepoints. The signal to background ratio and Z′ values were calculated against cells grown in the absence of scAAV-Nluc transduction. The CV values (defined herein as (standard deviation/average)*100) for the four technical replicates were calculated for each condition.

A robust and stable signal (defined here as >5,000 RLUs, or Relative Light Units) was obtained for all dilutions tested at both 6-hour and 24-hour timepoints (FIGS. 3A and E).

After 6 hours post transduction, the CV values for the first four dilutions of AAV-BrightLuc vector tested were predominantly under 10%, or predominantly under 20% for the last dilution tested (1/30,000); and predominantly under 80% for the non-transduced cells control (FIG. 3C). Signal to background ratio (S:B, defined as the fold difference between cells transduced with reporter vector (AAV-eGFP or AAV-BrightLuc) and cells not transduced with reporter vector) was typically greater than 100-fold for all conditions tested (FIG. 3B), and despite the higher CV of non-transduced cells and the 1/30,000 dilution, Z′ values remained over 0.5 for predominantly all dilutions other than the control (FIG. 3D). At 24 hours, the S:B ratio obtained was also greater than 100× in all conditions tested (FIG. 3F), while CVs were below 10% yielding excellent Z′ values of greater than 0.8 for predominantly all dilutions other than the control (FIG. 3H). Taken together, these results indicated that a robust assay signal could be obtained at 6 hours or 24 hours after transduction with MOIs (vg:cell) of 8333:1-0.83:1.

### Example 3—Testing of Additional Dilutions of scAAV-Nluc at 3, 6 and 24 Hours with Different Cell Numbers Per Well

Several dilutions of scAAV-Nluc revealed robust signal after 6 hours of transduction. To explore the limits of this assay signal, the experiment was repeated with both 1/3,000 and 1/30,000 dilutions of AAV-BrightLuc and included a further 1/300,000 dilution. In addition, to characterise the impact of the cell number (and thus the available AAV association sites) on assay signal, 2.5×10⁴, 5×10⁴, and 10×10⁴ cells/well were tested. Finally, the luminescent signal was measured at 3 hours, 6 hours and 24 hours. A summary of the resulting assay conditions tested is provided in Table 1 below.

TABLE 1
Summary of the assay conditions used to evaluate the luminescent assay signal over 3, 6 and 24 hours while
varying the amount of vector used and cells/well (an MOI of zero means that the MOI was less than 1).

| | | dil at | | at | | at | | | sample at | sample |
|---|---|---|---|---|---|---|---|---|---|---|
| | dil 2 | neutralisation | | neutralisation | on cells | neutralisation | on cells | MOI | neutralisation | on cells |
| reporter | (1:x) | (1:x) | cells/well | (vg/mL) | (vg/mL) | (vg) | (vg) | (vg/cell) | (%) | (%) |
| scAAV-eGFP | 750 | 10938 | 11700 | 5.8E+07 | 5.8E+07 | 1.0E+07 | 2.9E+06 | 250 | 1 | 1 |
| (REFERENCE) | | | | | | | | | | |
| scAAV-NLuc | NA | 3000 | 100000 | NA | 1.7E+07 | NA | 8.3E+05 | 8 | 10 | 10 |
| scAAV-NLuc | NA | 30000 | 100000 | NA | 1.7E+06 | NA | 8.3E+04 | 1 | 10 | 10 |
| scAAV-NLuc | NA | 300000 | 100000 | NA | 1.7E+05 | NA | 8.3E+03 | 0.83 | 10 | 10 |
| scAAV-NLuc | NA | 3000 | 50000 | NA | 1.7E+07 | NA | 8.3E+05 | 17 | 10 | 10 |
| scAAV-NLuc | NA | 30000 | 50000 | NA | 1.7E+06 | NA | 8.3E+04 | 2 | 10 | 10 |
| scAAV-NLuc | NA | 300000 | 50000 | NA | 1.7E+05 | NA | 8.3E+03 | 0.83 | 10 | 10 |
| scAAV-NLuc | NA | 3000 | 25000 | NA | 1.7E+07 | NA | 8.3E+05 | 33 | 10 | 10 |
| scAAV-NLuc | NA | 30000 | 25000 | NA | 1.7E+06 | NA | 8.3E+04 | 3 | 10 | 10 |
| scAAV-NLuc | NA | 300000 | 25000 | NA | 1.7E+05 | NA | 8.3E+03 | 0.83 | 10 | 10 |

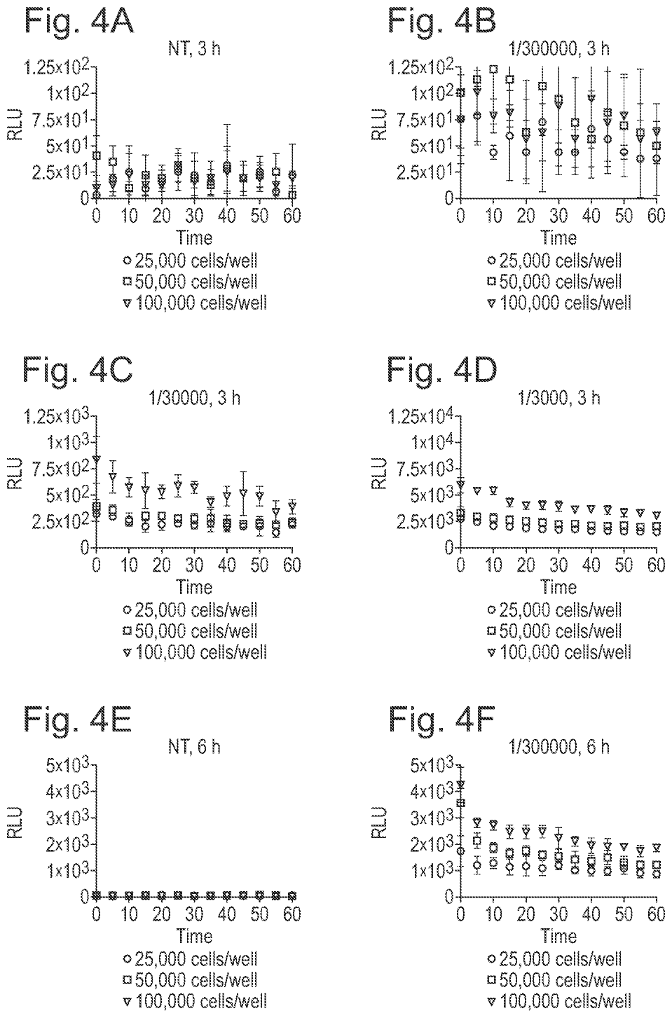

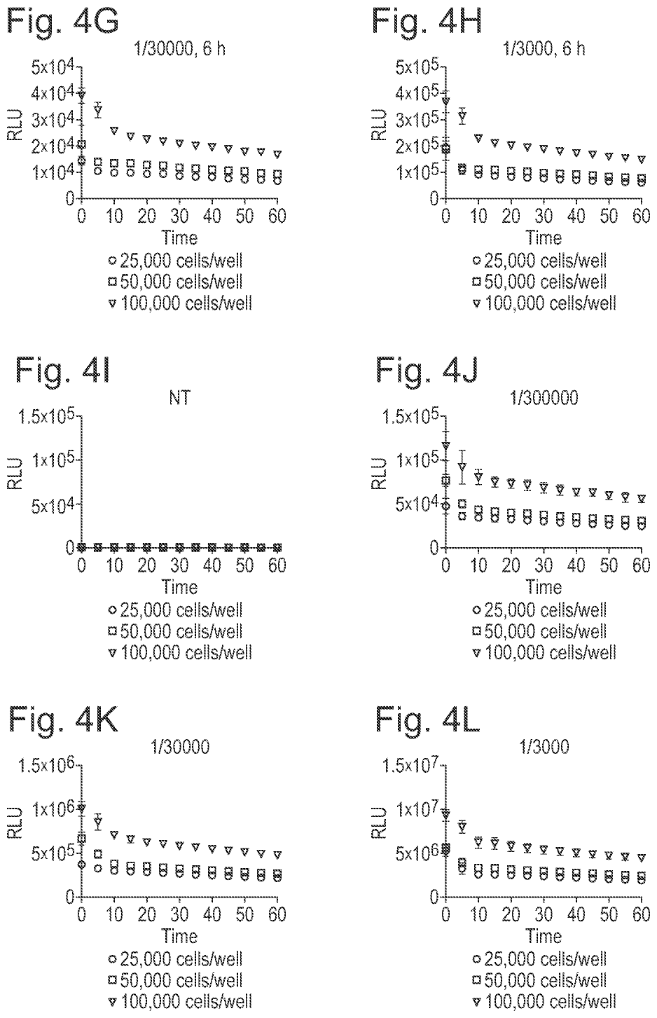

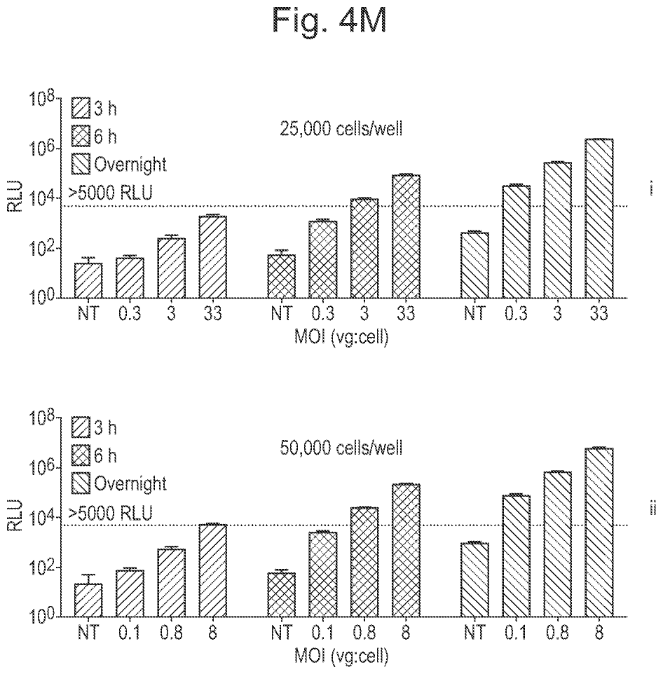

A robust assay signal (>5,000 RLUs) was obtained for all conditions tested at 24 hours after transduction (FIG. 4J-L). At 6 hours after transduction, only 1/3,000 and 1/30,000 dilutions of the AAV-BrightLuc vector yielded >5,000 RLUs (FIG. 4F-H), while at 3 hours, none of the assay conditions satisfied this criterion (FIG. 4B-D). A separate experiment with 100,000 cells per well transduced with AAV-BrightLuc for 3 hours at an MOI of 8 yielded >5,000 RLUs (FIG. 4M) at a Z′value of >0.5. Consistently, use of 100,000 cells/well yielded a nearly 2-fold increase in luminescent signal over both 50,000 and 25,000 cells, despite the lower MOI (Table 1). Without being bound by any theory, the present inventors speculate that one possible reason for this is that—since each cell offers several sites of binding/entry for every vector particle applied to them—a higher number of cells can mean that more (if not all) of the vector particles applied have a chance to enter the cell. Accordingly, at low MOI, this presents the possible scenario where every AAV vector applied to cells is likely to transduce. This means that any neutralisation event (prevention of AAV association with a cell), will be more likely to be detected.

Taken together, these results indicate that use of 100,000 cells/well with a minimum of 1/30,000 dilution of AAV-BrightLuc vector yields a robust assay signal within 6 hours of transduction.

### Example 4—Testing of scAAV-Nluc Neutralisation Using IVIG

In the cTIA, one of the positive controls consists a 1:2000 dilution of IVIG in the presence of 1% negative (“1%-ve”) plasma, defined herein as 1% negative plasma with normal growth media (DMEM) as the diluent. This mix is incubated with 5.8×10⁷ vg/ml of AAV-eGFP over 1 hour during neutralisation. Subsequently, 50 μl (2.9×10⁶ vg) of this mix is transferred to 1.2×10⁴ HEK293T cells growing on a 96-well dish, resulting in an MOI (vg:cells) of 250:1. In a successful run, this positive control yields an eGFP signal equivalent to less than 1% of the un-neutralised negative control (maximum signal), which consists of the same conditions but omits IVIG.

To determine whether the conditions established in the Materials and Methods section above produce a result equivalent to the cTIA, the level of inhibition with a 1:2000 dilution of IVIG was tested. In this experiment, neutralisation was carried out over 1 hour in a 50 μl volume in the presence of 1:2000 dilution of IVIG and 1%-ve plasma. In addition to the untransduced control, the amount of AAV-BrightLuc vector at this step was either 1/1,500, 1/15,000 or 1/150,000, which corresponds to 1.7×10⁷, 1.7×10⁶ or 1.7×10⁵ vg/ml respectively. 25 μl of this material was mixed with 25 μl of cell suspension containing 1×10⁵ cells in phenol-red free DMEM and 1%-ve plasma, yielding MOIs of 8.3, 0.83 and 0.083 to 1 respectively for each amount of AAV-BrightLuc reporter vector used. The assay was completed by supplementing with 50 μl of the Nano-Glo® reagent and reading the plate after 20 minutes of luminescent signal development.

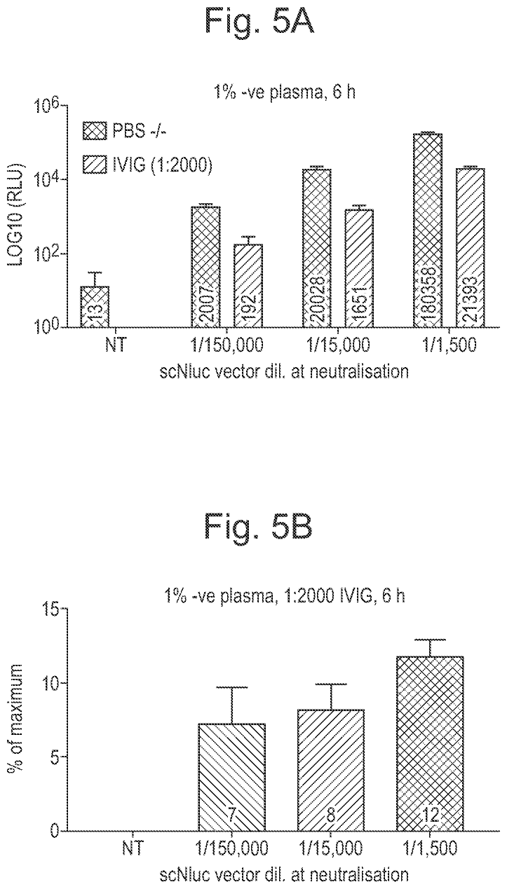

A robust signal of greater than 1×10⁴ RLUs was obtained with both 1/3,000 and 1/30,000 dilutions of vectors (on cells). A weaker (1×10³ RLU) signal was obtained for the 1/300,000 dilution of the AAV-BrightLuc reporter vector (FIG. 5A, designated “scNLuc”). The non-transduced control yielded an average background of 50 RLUs/well. The 1:2000 dilution of IVIG did not produce complete inhibition in any of the conditions tested, with 1/150,000 dilution of AAV-BrightLuc still yielding about 100 RLU or ^{˜}2× above non-transduced background.

For each condition tested, the percentage of maximum values were calculated by dividing the un-inhibited transduction signal by the matching IVIG-neutralised values. The remaining luminescence signal for 1/3,000, 1/30,000 and 1/300,000 dilutions of AAV-BrightLuc vector were 12.5%, 8% and 7% of maximum respectively (FIG. 5B).

Surprisingly, it was found that reduction of the AAV-BrightLuc vector amount used at neutralisation over 3 logs did not reduce the signal to below 1% of maximum, as achieved with the cTIA using eGFP. This is despite the amount of vector used at neutralisation and subsequent MOI (vg:cell) being similar or much lower than that of cTIA. Interestingly, the amount of inhibition achieved seems to plateau to about 93% (luminescent signal at 7% of maximum) instead of progressing to 100% as the amount of vector used is reduced. This indicates that for a fixed amount of IVIG (i.e. 1:2000 dilution) any additional reductions in the amount of vector used at the neutralisation step are unlikely to lead to 100% inhibition. Importantly, the inhibition plateau observed is not due to the inhibition signal equaling that of non-transduced controls (i.e. the assay bottoming out), as the signal produced by the IVIG-inhibited 1/300,000 dilution of AAV-BrightLuc is still roughly ^{˜}15 fold higher than that of non-transduced controls (FIG. 5A).

Taken together, these results indicate that there is no advantage to further reductions beyond 1/15,000 dilution (1.7×10⁶ vg/ml at neutralisation, MOI 0.83:1) in the amount of AAV-BrightLuc vector, which is the lowest amount of vector that still produces a robust assay signal.

### Example 5—Determining the Neutralising Titre of IVIG Using scAAV-Nluc

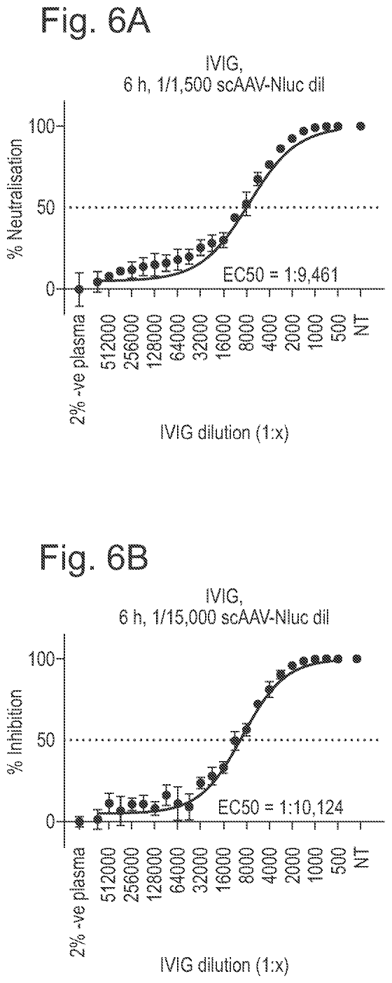

A 1:2000 dilution of IVIG did not result in greater than 99% inhibition when incubated with very low dilutions of AAV-BrightLuc (at neutralisation: 1/150,000 or 1.7×10⁵ vg/ml). Further dilutions are not optimal since the luciferase signal produced is less than 1×10⁴ RLU. To determine the impact of these conditions on the apparent neutralising titre of IVIG, 100,000 cells/well (as described in Example 3) were incubated with either 1/30,000 (MOI 0.83:1) or 1/3,000 (MOI 8.3:1) of AAV-BrightLuc over 6 hours after a 1 hour neutralisation with IVIG dilutions in 2% negative plasma, during which the vector concentration was respectively 1.7×10⁶ and 1.7×10⁷ vg/ml. The amount of negative plasma applied to cells during infection is determined by the starting dilution of a sample being tested. For example, a 1:100 starting dilution in cTIA results in 1% negative plasma applied to cells during the 2-day transduction step. At this stage of the assay development, a 2% negative plasma background was chosen so that eventual sample testing could provide enhanced sample stratification by beginning with a 1:50 dilution. Starting at 1:500, a further 21 IVIG dilutions were generated using a 1.41 dilution factor. Positive controls consisted of untransduced cells (NT), while the negative control consisted of AAV-BrightLuc vector incubated for 1 hour and transducing for 6 hours in the presence of 2% negative plasma. The resulting luminescence values were normalised to the signal of negative (0% inhibition) and positive (100% inhibition) controls before a four-parameter variable slope model was used to estimate the resulting EC50s. The neutralising titre of IVIG established with cTIA is 1:10,000. In this experiment, the EC50 titre obtained with both vector dilutions was 1:10,124 and 1:9,461 with 1/15,000 (FIG. 6B) and 1/1,500 (FIG. 6A) vector dilution at neutralisation respectively. At 1:2000 IVIG dilution, inhibition was 96 and 93% for 1/15,000 and 1/1,500 AAV-BrightLuc vector dilution at neutralisation respectively. Greater than 99% inhibition was only obtained at 1:1000 IVIG dilution with both amounts of AAV-BrightLuc vector used.

These results indicate that a log difference in the amount of reporter vector used during the transduction inhibition assay has minimal impact on the overall measurement of the neutralising titre. Furthermore, 1:1000 IVIG dilution is required before >99% inhibition of luminescent signal is obtained. Together with results from Example 4, these outcomes indicate that with these assay conditions, the neutralising titre of plasma samples obtained may be 2-fold less than that obtained with cTIA.

### Example 6—Comparing 6 Hour rTIA (Rapid TIA) with cTIA Neutralising Titres

The best assay conditions for sample testing have been determined to include the use of 1×10⁵ cells/well (Example 3) and a vector dilution at neutralisation between 1/1,500 (1.7×10⁷ vg/ml) and 1/15,000 (1.7×10⁶ vg/ml) (Example 5). At both these amounts of AAV-BrightLuc vector, 1:1000 IVIG resulted in 99% inhibition of the luminescent signal, justifying its use as positive control in all future experiments (Example 5).

To further compare the two methods, the discrete neutralising titre and calculated EC50s of samples tested with cTIA was determined by using the conditions established in the above Examples, but with the following modifications:

1. 1 hour neutralisation of a 1/6,000 dilution (8.3×10⁶ vg/ml) of scAAV-Nluc vector in the presence of 50% negative plasma or sample (1:2 starting dilution of sample)
2. To enable titering to begin with a 1:2 dilution of a test sample, a final 10% negative plasma applied to cells (using the methodology set out above)
3. 6 hours of transduction on 1.5×10⁵ cells/well—an increase in cells/well of 50% to ensure robust luminescent signal (>1×10⁴ RLU)

Starting at 1:2 dilution of a test sample in negative plasma, a further 11 dilutions were generated using a 2-fold dilution factor. A 1:1000 dilution of IVIG at neutralisation was used as positive control, while the negative control consisted of AAV-BrightLuc vector incubated for 1 hour and applied to cells (transduction) for 6 hours in the presence of 10% negative plasma. The resulting luminescence values were normalised to the signal of negative (0% inhibition) and positive (100% inhibition) controls before a four-parameter variable slope model was used to estimate the neutralising titre. The term “EC50” is used here interchangeably with the term “T150” (meaning transduction inhibition 50%) to reflect the estimation of inhibition titre of a sample using this method.

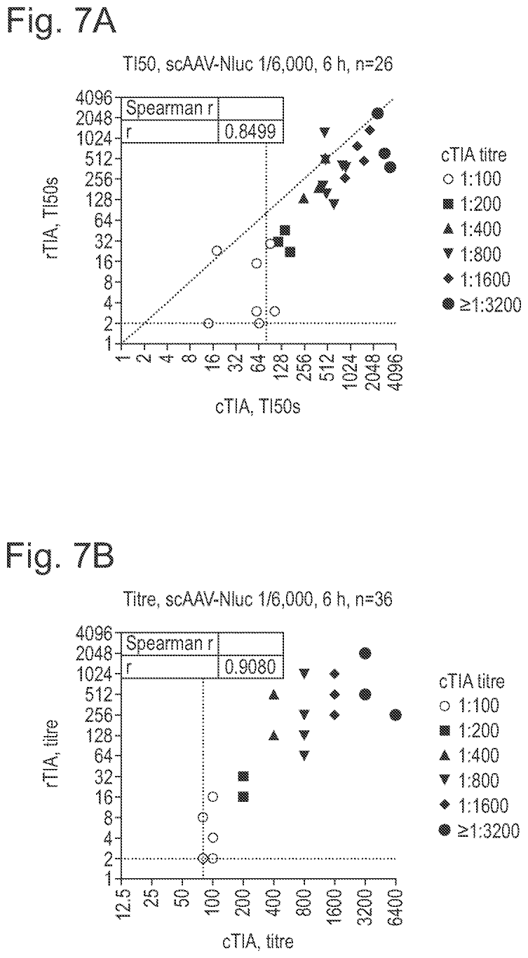

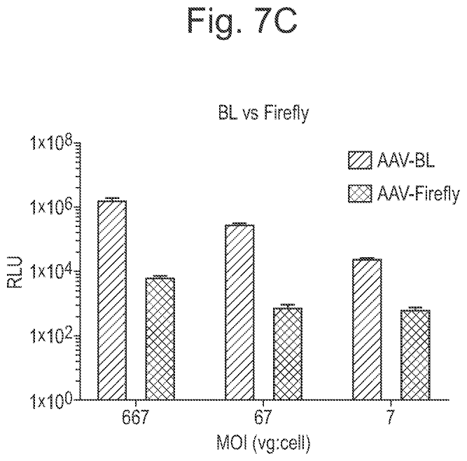

36 normal healthy human plasma samples obtained from LGC and TCS (commercial providers) were assayed with the protocol conditions described in this section. To enable comparison, the existing cTIA titres for these samples were re-analysed to estimate their TI50/EC50 values. Because some cTIA assays were carried out with only 4 dilutions (1/100, 200, 300 and 400), T150's were not generated from all available samples. Therefore, 26 and 36 comparison points could be generated for TI50 (FIG. 7A) and titre (FIG. 7B) comparison respectively.

Comparison of rTIA and cTIA results was carried out by computing the Spearman rank correlation test for both T150s and discrete titres. For each correlation plot, the shape of each data point indicates the discrete titre obtained in cTIA. Correlation coefficients of 0.85 (FIG. 7A) and 0.91 (FIG. 7B) were obtained for T150s and discrete titre comparisons respectively, indicating that sample ranking is similar between rTIA and cTIA.

A 3-fold drop in rTIA T150s was obtained for all samples over 1/400 by comparison with cTIA. Surprisingly, it was found that this decrease in apparent neutralising titre was exacerbated (up to 10-fold) for samples 1/400. A similar outcome was observed when discrete titres were compared.

A better correlation between rTIA and cTIA was obtained when discrete titres were used (0.91 vs 0.85 for TI50s) likely because of the expanded number of comparison points available (26 for T150s vs 36 for discrete titres). Together, the excellent correlation between cTIA and rTIA indicates that both assays will likely rank patients similarly. Given this excellent correlation between rTIA and cTIA and the availability of samples from patients that have already been screened and dosed based on the latter, it is clear that the method of the invention provides comparably accurate results to conventional methods in a shorter time-frame.

### Example 7—Development and Testing of a 16 Hour (Overnight) Transduction Inhibition Assay Protocol

In addition to the 6 hour rTIA protocol, an overnight (16 hour) protocol using the same AAV-BrightLuc vector was designed to ensure maximum flexibility with sample testing in the clinical setting. Results from Example 6 revealed that the greatest discrepancies in neutralising titre were obtained with a cTIA titre of ≤1:400. Therefore, instead of IVIG which has an apparent titre of 1/10,000 with both cTIA and rTIA, plasma sample TCS93 was chosen for testing. The 6 hour protocol used in Example 6 (described in the Materials & Methods section) was used without further modifications beyond the transduction time component. Two assay plates were set up, to be completed at 6 hours and 16 hours post transduction. T150s were calculated as previously described (see Example 6).

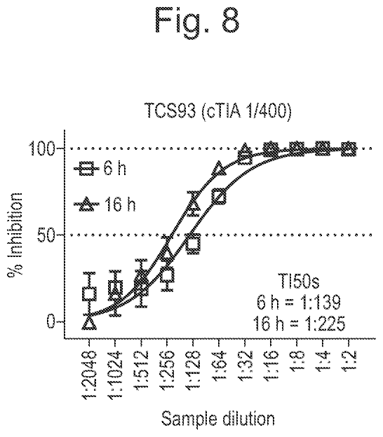

For this sample, a TI50 of 1:139 and 1:225 was obtained at 6 hours and 16 hours respectively, yielding a ^{˜}40% increase in the apparent neutralising titre (FIG. 8). In Example 6, the same sample presented with a TI50 of 1:128 with the 6 hour protocol, suggesting that the 6 hour and 16 hour protocols may produce different TI50s. While this outcome could be the result of extended transduction, another possible explanation is simple assay variation.

### Example 8—Expanded Testing of the Overnight (16 h) Protocol

Preliminary testing described in Example 7 established that evaporation reduced the resulting luminescent signal in the outer edges of the 96-well assay plate, likely by altering cell growth conditions overnight. To circumvent this problem, the outer edges of the plate were not included in the assay plate layout. The final 6 hour rTIA includes 1.5×10⁵ cells/well cultured over 6 hours of transduction. The same number of cells may grow to contact inhibition overnight, resulting in possible distortion of the assay signal which is dependent on optimal cell growth and by extension, gene expression. To prevent issues related to cell growth, the number of cells/well was reduced to 1×10⁵.

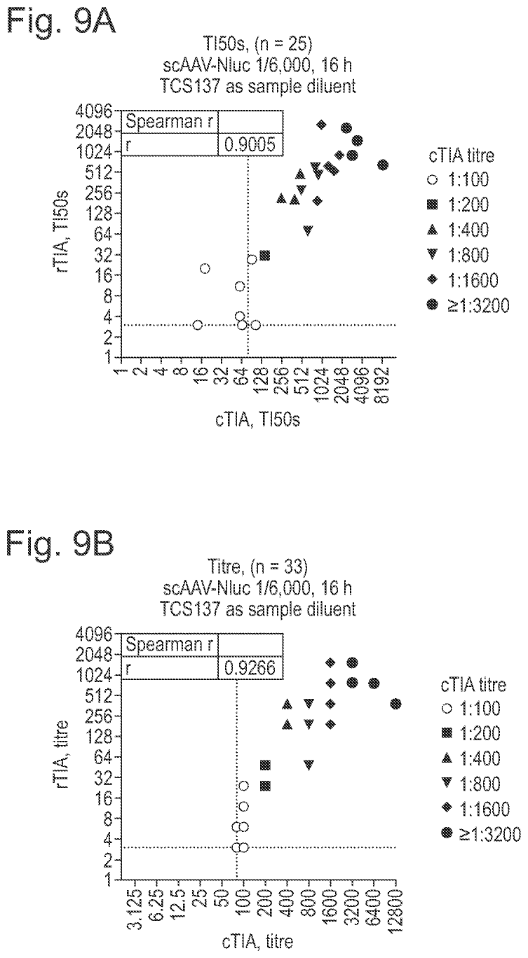

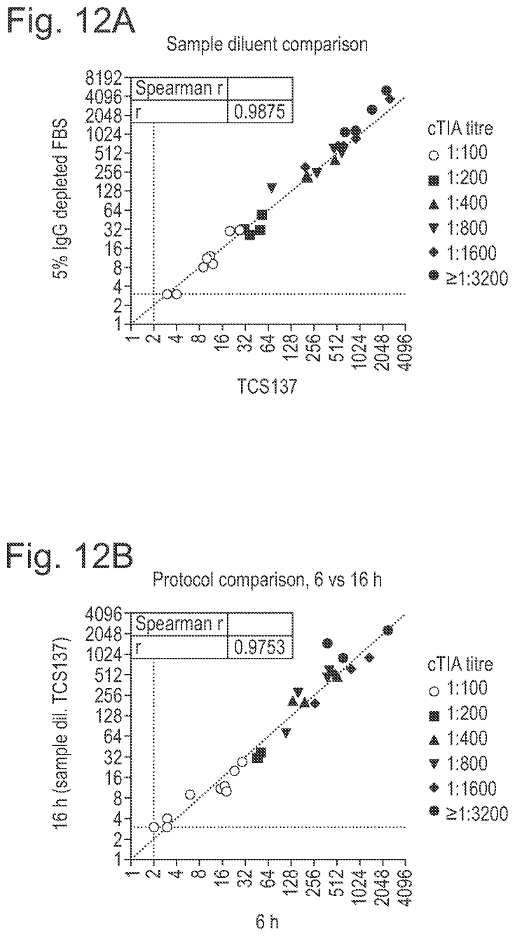

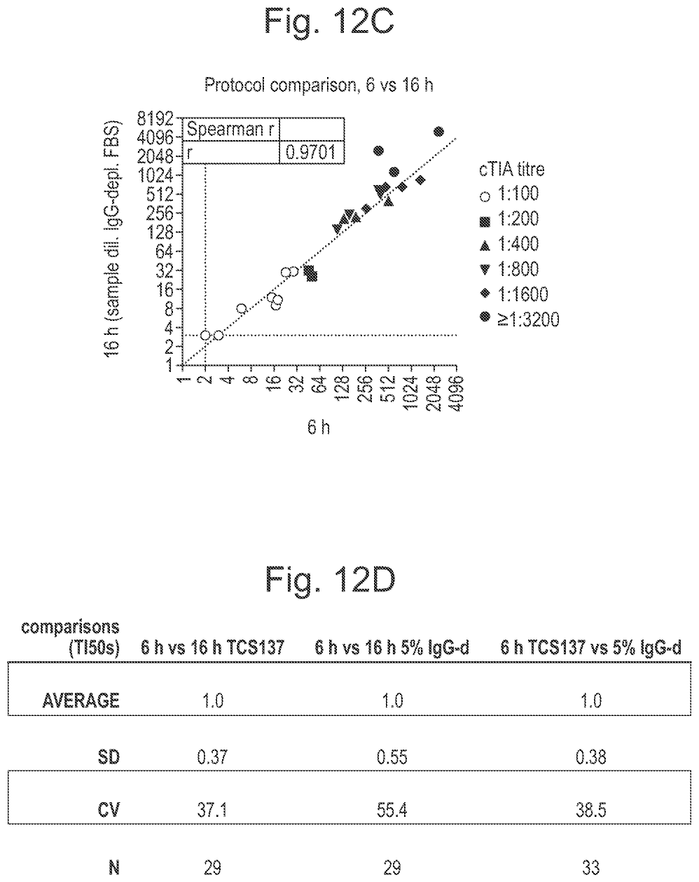

All other assay conditions were kept identical to the 6 hour protocol described in the above Examples. Testing of available samples produced 25 and 33 comparison points (cTIA vs rTIA) for T150s and discrete titres respectively. Sample ranking using the 16 hour protocol was similar to cTIA (Spearman rank coefficient of 0.90 and 0.93 for T150s and discrete titres respectively, FIGS. 9A and 9B). Sample ranking between the 6 hour and 16 hour protocols was nearly identical (FIG. 12B, and further elaborated in Example 10). The fold changes between the 16 hour rTIA protocol and the GFP-based cTIA were also about 3-fold for samples of >1/400 titre (by cTIA) increasing up to 10-fold for the remaining samples of ≤1:400. However, on average no difference in titre was found when the T150s of the 6 hour and 16 hour protocols were compared (average fold difference 1.0 with an SD of 0.4 over 29 samples, FIG. 12D). Taken together, these results indicate that the 16 hour protocol performs very similarly to the 6 hour described in the above Examples.

### Example 9—Standardising of Sample Diluent: Comparison of IgG-Depleted FBS and AAV-NAF Negative Healthy Human Plasma

Commercial companion diagnostic assays require the standardisation of all reagents used. The sample diluent used in Examples 2 to 8 above was plasma from a healthy human donor. Because of the limited availability and production traceability of human plasma samples, a different and more commonly available assay diluent was sought. HEK293T cells are routinely maintained in DMEM supplemented with 10% foetal bovine serum (FBS). This component of the growth media is available in protein-G IgG depleted form with an advertised IgG concentration of less than 5 ug/mL (Gibco catalogue 16250086). In the present experiment, the use of IgG-depleted FBS as sample diluent was investigated.

Sample TCS85, an abundant test plasma sample, was diluted as previously described (Materials & Methods above) using negative plasma, and either a 50% or 10% dilution thereof in phenol red-free DMEM. In parallel, the same sample was diluted in IgG-depleted FBS, as well as a 50% or 10% dilution thereof in phenol red-free DMEM. Because of sample treatment, the resulting concentration of diluent (negative plasmas or IgG-depleted FBS) in the assay plate is 10%, 5% or 1% for neat, 50%, and 10% compositions of the sample diluent. The assay was then carried out using the 16 hour protocol described above in Example 8.

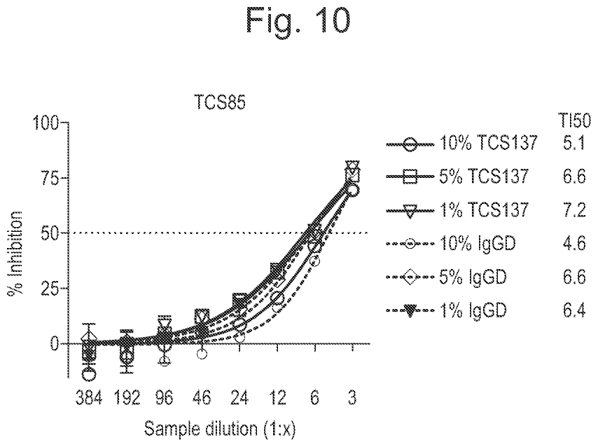

The T150s obtained with IgG-depleted FBS are very similar to those obtained with negative plasma (“TCS137,” FIG. 10). Use of neat negative plasma or IgG depleted FBS produced a lower titre when compared to 50% and 10% dilutions. The 50% and 10% dilutions of the assay diluent provide any additional shift (>20%) in the T150 values. Taken together, this result establishes the equivalency between negative plasma and IgG depleted FBS and indicates that a 50% dilution of FBS is likely to provide the best assay sensitivity.

### Example 10—Re-Testing of Healthy Plasma Samples Using 50% IgG-Depleted FBS as Sample Diluent

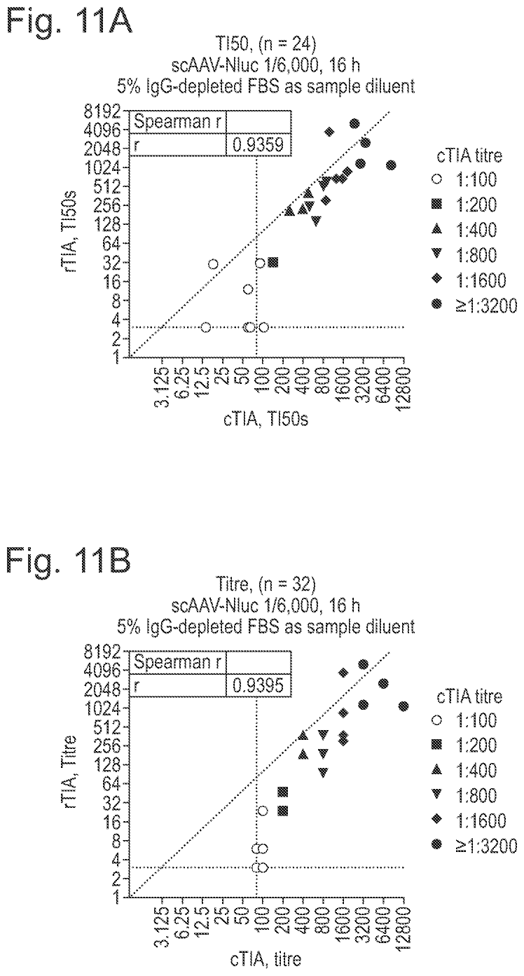

The 16 hour protocol described above was used to retest healthy plasma samples. Testing of available samples produced 24 and 32 comparison points (AAV-eGFP vs AAV-BrightLuc) for T150s and discrete titres respectively. Sample ranking using IgG-depleted FBS as sample diluent was similar to cTIA (Spearman rank coefficient of 0.94 for both T150s and discrete titres, FIGS. 11A and 11B). Sample ranking between the 6 hour and 16 hour protocols was nearly identical (FIG. 12C). The fold changes between the 16 hour rTIA protocol and cTIA remained at about 3-fold for samples of >1/400 titre (by cTIA) increasing up to 10-fold for the remaining samples of ≤1:400. However, on average no difference in titre was found when the T150s of 6 hour and 16 hour protocols were compared (average fold difference 1.0 with an SD of 0.55 over 29 samples, FIG. 12D).

Finally, the 16 hour protocol results from both negative plasma and IgG-depleted FBS samples were compared. A Spearman rank correlation of 0.99 was obtained indicating strong agreement between the T150 values produced by these protocols (FIG. 12A). Furthermore, there was no difference in the average value of the T150s (average fold change of 1.0 with an SD of 0.39) over 33 comparison points (FIG. 12D).

Taken together, these results indicate that the 16 hour protocol using 50% IgG-depleted FBS as sample diluent performs very similarly to the 6 hour protocol as described in Example 2 to Example 6 and the 16 hour protocol described in Example 8.

### Example 11—Examining the Effect of Plasmapheresis on Antibody and NAb Titre

Double filtration plasmapheresis (DFPP) is a routine method for removing antibodies from circulation and is conventionally used in patients prior to undergoing transplantation surgery. Biobank plasma samples from 36 patients who had undergone up to 5 rounds of DFPP prior to kidney transplant were retrospectively analysed. Pre- and post-cycle plasma samples were tested for anti-AAV antibodies using both ELISA and a GFP-based conventional TIA (cTIA) using the methodology described above.

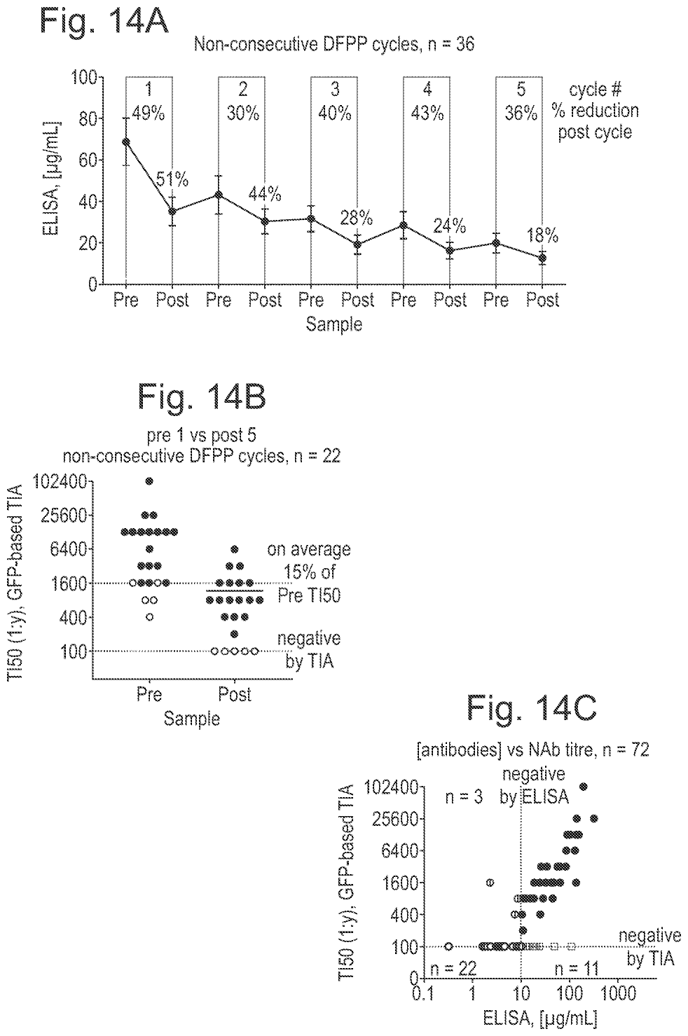

After each successive round of DFPP in transplant patients, the total and neutralising titres of AAV Ab decreased. A pattern of antibody re-synthesis and/or redistribution from tissues was observed between cycles, although subsequent rounds always enabled further reductions in antibody titre compared to baseline. ELISA analysis (FIG. 14A) confirms that regardless of the starting titre, there was a decrease and subsequent rise in antibody levels after each DFPP cycle. The mean trend of all data shows the largest reduction in anti-AAV antibodies after the first cycle which continues with each successive cycle to show an overall decline across all patients.

The GFP-based TIA (cTIA) found (FIG. 14B) a decrease in AAV NAbs for all patients following 5 cycles of DFPP by comparison with the titres pre-DFPP. In this assay, all titres at or below 1:100 are defined as negative for AAV NAbs, meaning that such a result would lead to a patient being considered eligible for gene therapy. It was found that 1:1600 was the highest starting (“pre”) titre to reach 1:100 following 5 cycles of DFPP (“post”). When considering seropositive patients with a starting titre of 1:1600, 62% of such patients that underwent DFPP achieved the titre required for eligibility.

### Example 12—Comparing the Results of TIA with ELISA

To demonstrate the utility of the AAV-NLuc based TIA assay in a disease relevant population, the neutralising titre was measured in 62 haemophilia B patient samples. Nanoluc-based TIA was carried out as described above, with a 16 h (overnight) immunocomplex formation step and 100,000 HEK239T cells in the 75 μL transduction reaction. An indirect ELISA to measure anti-AAV IgGs was carried out as follows: The capture substrate, an AAV viral vector particle, was coated onto the surface of a NUNC MaxiSorp 96-well plate overnight at +2 to +8° C. Following a wash step to remove any unbound AAV antigen, the plates were blocked. A further wash step to remove the blocking buffer was followed by the addition of controls and test samples.

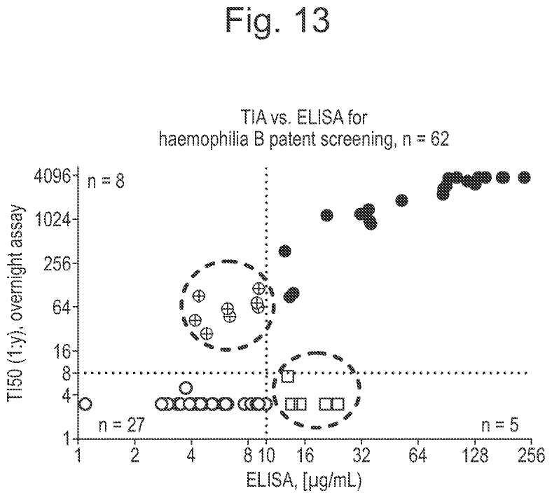

Following incubation, and washing away any unbound material, a polyclonal anti-human antibody conjugated to HRP was added. After incubation with the detection antibody and a further wash step, the substrate chromogen SIGMA FAST™, OPD (o-Phenylenediamine dihydrochloride) peroxidase was added to the wells. The reaction was stopped by addition of 3M HCl and the optical density was determined using a microplate reader set to 492 nm with a reference wavelength of 630 nm. When comparing the TIA and ELISA results, it was found that an ELISA cut-off of 10 μg/mL produces false-negative and false-positive rates of 16 and 27% respectively (FIG. 13).

Finally, a comparison (FIG. 14C) between the ELISA and the cTIA results showed a reduced ELISA accuracy by comparison with the TIA when determining eligibility to AAV gene therapy. In more detail, the ELISA methodology produces both false negatives and false positives. Without being bound by any theory, the present inventors speculate that ELISA methodology measures overall antibody content and not neutralising antibodies (NAb), in contrast to the TIA of the present invention which allows accurate determination of NAb titre.

### Example 13—Comparing the Effects of Consecutive Vs Non-Consecutive DFPP

The plasma samples tested in Example 11 above were obtained from patients who were undergoing DFPP prior to kidney transplantation. In that instance, the five cycles of DFPP were in most cases not carried out on successive days (deemed “consecutive”) but at longer intervals (deemed “non-consecutive”), usually with 48 hours or 72 hours elapsing between cycles, and occasionally more.

As shown in Example 11 above and FIG. 13, an accurate determination of NAb titre in the clinic requires TIA since ELISA methods are not sufficiently accurate to determine eligibility for gene therapy. To facilitate the rapid turnaround of NAb analysis required for daily apheresis cycles (deemed “consecutive”), the rapid TIA (rTIA/BL-TIA) of the invention has been developed, which uses a synthetic bright luciferase (NanoLuc®) and which (as shown in the examples above) can produce accurate results in 24 hours or less.

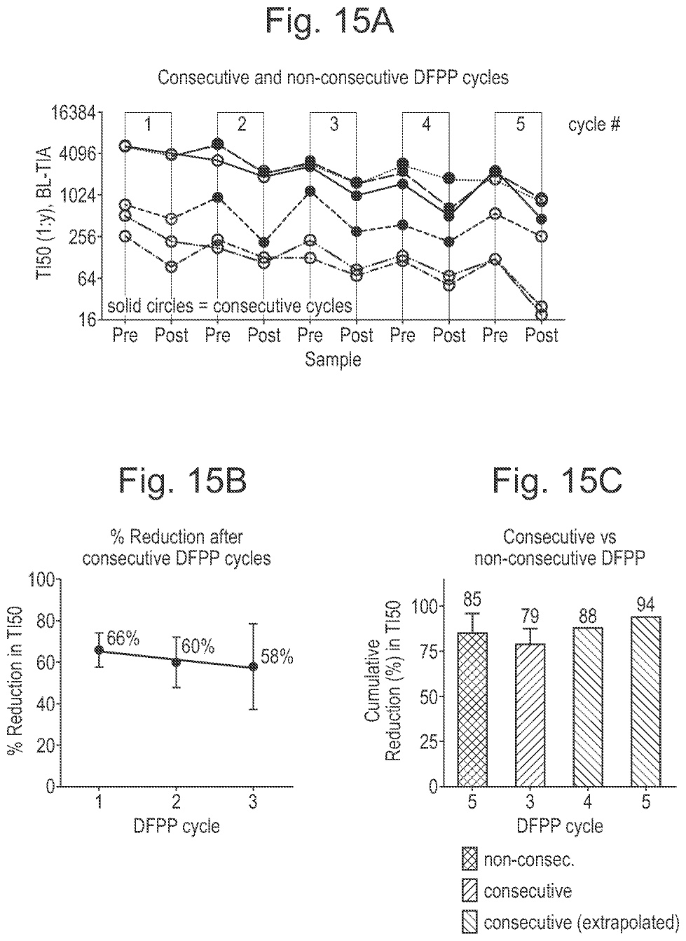

The BL-TIA assay of the invention was used to retest samples from six of the patients from Example 11 above, four of whom underwent 3 rounds of DFPP on successive days (“consecutive”). As shown in FIG. 15A, DFPP cycles 2, 3 and 4 or cycles 3, 4, and 5 were “consecutive” meaning that they were delivered on successive days. Thus, cycle 3 was delivered the day after cycle 2; and/or cycle 4 was delivered the day after cycle 3.

Analysis of samples from this “consecutive” administration of DFPP yielded a reduction in NAb titre (measured using the T150) of 66%, 60%, and 58% for the first, second and third cycles, respectively (FIG. 15B).

An analysis of the initial and final samples from these 6 patients found an average of 85% reduction in AAV NAb titre (FIG. 15C), which was in good agreement with the findings from Example 11 above, using the GFP-based TIA (FIG. 14B).

Analysing consecutive rounds of DFPP confirm that the first round is most efficient: 66% of AAV NAbs were removed on the first consecutive day with similar, but lower, percentages removed on subsequent days. Overall, 3 “consecutive” rounds of DFPP decreased AAV NAb titres by 79±9%.

To further understand the minimum number of cycles required to reduce AAV NAbs, regression analysis was performed, using average reductions obtained using three “consecutive” DFPP cycles (i.e. where each cycle is performed the day after the previous cycle). This analysis showed that four “consecutive” cycles of DFPP yield a similar NAb reduction to five “non-consecutive” cycles. Importantly, five “consecutive” cycles are predicted to reduce the starting AAV NAb titre by 94% (FIG. 15C) and thereby improve the potential reduction in AAV NAb titre.

Accordingly, using the TIA of the invention which crucially provides an accurate NAb titre result in substantially less than 24 hours and (as demonstrated above) can reliably do so after only 16 or even 6 hours of transduction, it can be seen that consecutive rounds of apheresis can reduce anti-AAV Nabs to levels aligned with clinical trial inclusion.

### Example 14—Comparing the Efficacy of the TIA Across AAV Serotypes

This TIA procedure described in the above examples can be used to compare the neutralising antibody (NAb) status of multiple (e.g. 96 or more) individual human plasma samples following incubation with any AAV serotype containing a vector genome encoding the Nanoluc luciferase from the CMV promoter.

All plasma samples are mixed 1:1 with a dilution of AAV vector (e.g. 1×10⁴, 1×10⁵, 1×10⁶ or more vg/mL of AAV-Nluc or AAV5-Nluc vector, or any other serotype of AAV, whether naturally-occurring or synthetic/engineered) so as to obtain a 50% plasma-vector mixture and incubated over a given amount of time (e.g. 1, 2 or 3 h) at 37° C. to enable complex formation between neutralizing factors in the plasma (e.g. antibodies) and AAV particles.

Subsequently, this material is diluted 1:5 in a cell suspension to obtain a mixture containing an appropriate number of cells (e.g. 25,000, 50,000 or 100,000 cells) of a relevant cell line or primary cells (e.g. HEK239T, HUH7s, liver primary cells etc).

After overnight incubation at 37° C., 5% CO₂, cells are lysed and NanoLuc expression quantified by mixing 1:1 with the NanoGlo reagent following the instructions provided with the reagent. The methods described above are used to categorize samples as either positive or negative for the presence of AAV neutralization.

The TIA of the invention provides a sufficient luminescent signal within the time-frame for many serotypes, including AAV5, which do not efficiently transduce a given cell line of interest (e.g. HEK293T). Thus the TIA of the invention provides acceptable assay quality and robustness parameters (e.g. Z′>0.5 and CVs <25%) even for those serotypes.

A key advantage of this method is the ability to compare transduction of serotypes with large variation in transduction efficiency in a short period of time (so as to support higher throughput) on the same cell line using typically low amounts of vector, such as those used by a efficiently transducing serotype. Low amounts of reporter vector (i.e. scAAV-NLuc) enable more sensitive TIAs and provide a more complete picture of seroprevalence. Taken together, this method enables comparison of seroprevalence by conducting the experiment under identical (amount of vector, time of incubation, cell type), high-throughput, and sensitive conditions.

### Sequence Listing Table

| SEQ | |
|---|---|
| ID NO. | Sequence description/derivation |
| 1 | Amino acid sequence of a firefly luciferase derived from |
| | Photinus pyralis (UniProt database Q27758) |
| 2 | Amino acid sequence of a synthetic luciferase commercially |
| | named “NanoLuc” (Promega); derived from Oplophorus |
| | gracilirostris |
| 3 | Amino acid sequence of a synthetic luciferase commercially |
| | named “TurboLuc” (Thermo Scientific); derived from the |
| | marine copepod Metridia pacifica luciferase family |
| 4 | Amino acid sequence of a synthetic luciferase commercially |
| | named “Lucia” (InvivoGen); derived from planktonic copepods |
| 5 | Amino acid sequence of an AAV capsid used in “scAAV-Nluc” |
| 6 | Recombinant AAV capsid protein, SEQ ID NO: 31 from WO |
| | 2013/029030 |
| 7 | Recombinant AAV capsid protein, SEQ ID NO: 46 from WO |
| | 2013/029030 |
| 8 | Recombinant AAV capsid protein, SEQ ID NO: 47 from WO |
| | 2013/029030 |
| 9 | Recombinant AAV capsid protein, SEQ ID NO: 54 from WO |
| | 2013/029030 |
| 10 | Recombinant AAV capsid protein, SEQ ID NO: 56 from WO |
| | 2013/029030 |
| 11 | An enzyme having IgG cysteine protease activity |
| 12 | An enzyme having IgG cysteine protease activity |

SEQ ID NO: 1
MEDAKNIKKGPAPFYPLEDGTAGEQLHKAMKRYALVPGTIAFTDAHIEVNITYAEYFEMSVRLAEAMKRYGLNTN
HRIVVCSENSLQFFMPVLGALFIGVAVAPANDIYNERELLNSMNISQPTVVFVSKKGLQKILNVQKKLPIIQKIIIMDS
KTDYQGFQSMYTFVTSHLPPGFNEYDFVPESFDRDKTIALIMNSSGSTGSPKGVALPHRTACVRFSHARDPIFGNQI
IPDTAILSVVPFHHGFGMFTTLGYLICGFRVVLMYRFEEELFLRSLQDYKIQSALLVPTLFSFFAKSTLIDKYDLSNLHEI
ASGGAPLSKEVGEAVAKRFHLPGIRQGYGLTETTSAILITPEGDDKPGAVGKVVPFFEAKVVDLDTGKTLGVNQRGE
LCVRGPMIMSGYVNDPEATNALIDKDGWLHSGDIAYWDEDEHFFIVDRLKSLIKYKGCQVAPAELESILLQHPNIFD
AGVAGLPGDDAGELPAAVVVLEHGKTMTEKEIVDYVASQVTTAKKLRGGVVFVDEVPKGLTGKLDARKIREILIKAK
KGGKSKL
SEQ ID NO: 2
MVFTLEDFVGDWRQTAGYNLDQVLEQGGVSSLFQNLGVSVTPIQRIVLSGENGLKIDIHVIIPYEGLSGDQMGQIE
KIFKVVYPVDDHHFKVILHYGTLVIDGVTPNMIDYFGRPYEGIAVFDGKKITVTGTLWNGNKIIDERLINPDGSLLFRV
TINGVTGWRLCERILA
SEQ ID NO: 3
MEAEAERGKLPGKKLPLEVLIELEANARKAGCTRGCLICLSKIKCTAKMKKYIPGRCADYGGDKKTGQAGIVGAIVDI
PEISGFKEMEPMEQFIAQVDRCADCTTGCLKGLANVKCSDLLKKWLPGRCATFADKIQSEVDNIKGLAGD
SEQ ID NO: 4
MEIKVLFALICIAVAEAKPTEINEDLNIAAVASNFATTDLETDLFTNWETMNVISTDTEQVNTDADRGKLPGKKLPP
DVLRELEANARRAGCTRGCLICLSHIKCTPKMKKFIPGRCHTYEGEKESAQGGIGEAIVDIPEIPGFKDKEPLDQFIAQ
VDLCADCTTGCLKGLANVQCSDLLKKWLPQRCTTFASKIQGRVDKIKGLAGDR
SEQ ID NO: 5
MAADGYLPDWLEDNLSEGIREWWALKPGAPKPKANQQKQDDGRGLVLPGYKYLGPFNGLDKGEPVNAADAAA
LEHDKAYDQQLQAGDNPYLRYNHADAEFQERLQEDTSFGGNLGRAVFQAKKRVLEPLGLVEEGAKTAPGKKRPV
DQSPQEPDSSSGVGKSGKQPARKRLNFGQTGDSESVPDPQPLGEPPAAPTSLGSNTMASGGGAPMADNNEGAD
GVGNSSGNWHCDSQWLGDRVITTSTRTWALPTYNNHLYKQISSQSGASNDNHYFGYSTPWGYFDFNRFHCHFS
PRDWQRLINNNWGFRPKKLSFKLFNIQVKEVTQNDGTTTIANNLTSTVQVFTDSEYQLPYVLGSAHQGCLPPFPA
DVFMVPQYGYLTLNNGSQAVGRSSFYCLEYFPSQMLRTGNNFQFSYTFEDVPFHSSYAHSQSLDRLMNPLIDQYLY
YLNRTQGTTSGTTNQSRLLFSQAGPQSMSLQARNWLPGPCYRQQRLSKTANDNNNSNFPWTAASKYHLNGRDS
LVNPGPAMASHKDDEEKFFPMHGNLIFGKEGTTASNAELDNVMITDEEEIRTTNPVATEQYGTVANNLQSSNTAP
TTRTVNDQGALPGMVWQDRDVYLQGPIWAKIPHTDGHFHPSPLMGGFGLKHPPPQIMIKNTPVPANPPTTFSP
AKFASFITQYSTGQVSVEIEWELQKENSKRWNPEIQYTSNYNKSVNVDFTVDTNGVYSEPRPIGTRYLTRNL
(= SEQ ID NO: 31 of WO 2013/029030)
SEQ ID NO: 6
MAADGYLPDWLEDNLSEGIREWWALQPGAPKPKANQQHQDNARGLVLPGYKYLGPGNGLDKGEPVNAADAA
ALEHDKAYDQQLKAGDNPYLKYNHADAEFQERLKEDTSFGGNLGRAVFQAKKRLLEPLGLVEEAAKTAPGKKRPV
DQSPQEPDSSSGVGKSGKQPARKRLNFGQTGDSESVPDPQPLGEPPAAPTSLGSNTMASGGGAPMADNNEGAD
GVGNSSGNWHCDSQWLGDRVITTSTRTWALPTYNNHLYKQISSQSGASNDNHYFGYSTPWGYFDFNRFHCHFS
PRDWQRLINNNWGFRPKKLSFKLFNIQVKEVTQNDGTTTIANNLTSTVQVFTDSEYQLPYVLGSAHQGCLPPFPA
DVFMVPQYGYLTLNNGSQAVGRSSFYCLEYFPSQMLRTGNNFQFSYTFEDVPFHSSYAHSQSLDRLMNPLIDQYLY
YLNRTQGTTSGTTNQSRLLFSQAGPQSMSLQARNWLPGPCYRQQRLSKTANDNNNSNFPWTAASKYHLNGRDS
LVNPGPAMASHKDDEEKFFPMHGNLIFGKEGTTASNAELDNVMITDEEEIRTTNPVATEQYGTVANNLQSSNTAP
TTRTVNDQGALPGMVWQDRDVYLQGPIWAKIPHTDGHFHPSPLMGGFGLKHPPPQIMIKNTPVPANPPTTFSP
AKFASFITQYSTGQVSVEIEWELQKENSKRWNPEIQYTSNYNKSVNVDFTVDTNGVYSEPRPIGTRYLTRPL
(= SEQ ID NO: 46 of WO 2013/029030)
SEQ ID NO: 7
MAADGYLPDWLEDNLSEGIREWWALQPGAPKPKANQQHQDNARGLVLPGYKYLGPGNGLDKGEPVNAADAA
ALEHDKAYDQQLKAGDNPYLKYNHADAEFQERLKEDTSFGGNLGRAVFQAKKRLLEPLGLVEEAAKTAPGKKRPV
DQSPQEPDSSSGVGKSGKQPARKRLNFGQTGDSESVPDPQPLGEPPAAPTSLGSNTMASGGGAPMADNNEGAD
GVGNSSGNWHCDSQWLGDRVITTSTRTWALPTYNNHLYKQISSQSGASNDNHYFGYSTPWGYFDFNRFHCHFS
PRDWQRLINNNWGFRPKKLSFKLFNIQVKEVTQNDGTTTIANNLTSTVQVFTDSEYQLPYVLGSAHQGCLPPFPA
DVFMVPQYGYLTLNNGSQAVGRSSFYCLEYFPSQMLRTGNNFQFSYTFEDVPFHSSYAHSQSLDRLMNPLIDQYLY
YLNRTQGTTSGTTNQSRLLFSQAGPQSMSLQARNWLPGPCYRQQRLSKTANDNNNSNFPWTAASKYHLNGRDS
LVNPGPAMASHKDDEEKFFPMHGNLIFGKEGTTASNAELDNVMITDEEEIRTTNPVATEQYGTVANNLQSSNTAP
TTRTVNDQGALPGMVWQDRDVYLQGPIWAKIPHTDGHFHPSPLMGGFGLKHPPPQIMIKNTPVPANPPTTFSP
AKFASFITQYSTGQVSVEIEWELQKENSKRWNPEIQYTSNYNKSVNVDFTVDTNGVYSEPRPIGTRYLTRPL
(= SEQ ID NO: 47 of WO 2013/029030)
SEQ ID NO: 8
MAADGYLPDWLEDNLSEGIREWWALQPGAPKPKANQQHQDNARGLVLPGYKYLGPGNGLDKGEPVNAADAA
ALEHDKAYDQQLKAGDNPYLKYNHADAEFQERLKEDTSFGGNLGRAVFQAKKRLLEPLGLVEEAAKTAPGKKRPV
DQSPQEPDSSSGVGKSGKQPARKRLNFGQTGDSESVPDPQPLGEPPAAPTSLGSNTMASGGGAPMADNNEGAD
GVGNSSGNWHCDSQWLGDRVITTSTRTWALPTYNNHLYKQISSQSGASNDNHYFGYSTPWGYFDFNRFHCHFS
PRDWQRLINNNWGFRPKKLSFKLFNIQVKEVTQNDGTTTIANNLTSTVQVFTDSEYQLPYVLGSAHQGCLPPFPA
DVFMVPQYGYLTLNNGSQAVGRSSFYCLEYFPSQMLRTGNNFQFSYTFEDVPFHSSYAHSQSLDRLMNPLIDQYLY
YLNRTQGTTSGTTNQSRLLFSQAGPQSMSLQARNWLPGPCYRQQRLSKTANDNNNSNFPWTAASKYHLNGRDS
LVNPGPAMASHKDDEEKFFPMHGNLIFGKEGTTASNAELDNVMITDEEEIRTTNPVATEQYGTVANNLQSSNTAP
TTRTVNDQGALPGMVWQDRDVYLQGPIWAKIPHTDGHFHPSPLMGGFGLKHPPPQIMIKNTPVPANPPTTFSP
AKFASFITQYSTGQVSVEIEWELQKENSKRWNPEIQYTSNYNKSVNVDFTVDTNGVYSEPRPIGTRYLTRPL
(= SEQ ID NO: 54 of WO 2013/029030)
SEQ ID NO: 9
MAADGYLPDWLEDNLSEGIREWWDLKPGAPKPKANQQKQDDGRGLVLPGYKYLGPFNGLDKGEPVNAADATA
LEHDKAYDQQLQAGDNPYLRYNHADAEFQERLQEDTSFGGNLGRAVFQAKKRVLEPLGLVEEGAKTAPGKKRPV
EPSPQRSPDSSTGIGKKGQQPARKRLNFGQTGDSESVPDPQPLGEPPAAPTSLGSNTMASGGGAPMADNNEGA
DGVGNSSGNWHCDSQWLGDRVITTSTRTWALPTYNNHLYKQISSASTGASNDNHYFGYSTPWGYFDFNRFHCH
FSPRDWQRLINNNWGFRPKRLNFKLFNIQVKEVTTNDGVTTIANNLTSTIQVFTDSEYQLPYVLGSAHQGCLPPFP
ADVFMIPQYGYLTLNNGSQAVGRSSFYCLEYFPSQMLRTGNNFQFSYTFEDVPFHSSYAHSQSLDRLMNPLIDQYL
YYLNRTQGTTSGTTNQSRLLFSQAGPQSMSLQARNWLPGPCYRQQRLSKTANDNNNSNFPWTAASKYHLNGRD
SLVNPGPAMASHKDDEEKFFPMHGNLIFGKEGTTASNAELDNVMITDEEEIRTTNPVATEQYGTVANNLQSSNTA
PTTRTVNDQGALPGMVWQDRDVYLQGPIWAKIPHTDGHFHPSPLMGGFGLKHPPPQILIKNTPVPANPSTTFSA
AKFASFITQYSTGQVSVEIEWELQKENSKRWNPEIQYTSNYYKSNNVEFAVNTEGVYSEPRPIGTRYLTRNL
(= SEQ ID NO: 56 of WO 2013/029030)
SEQ ID NO: 10
MAADGYLPDWLEDTLSEGIRQWWALKPGAPKPKANQQKQDDGRGLVLPGYKYLGPFNGLDKGEPVNAADAAA
LEHDKAYDQQLKAGDNPYLRYNHADAEFQERLQEDTSFGGNLGRAVFQAKKRVLEPLGLVEEGAKTAPGKKRPVE
PSPQRSPDSSTGIGKKGQQPARKRLNFGQTGDSESVPDPQPLGEPPAAPTSLGSNTMASGGGAPMADNNEGAD
GVGNSSGNWHCDSQWLGDRVITTSTRTWALPTYNNHLYKQISSASTGASNDNHYFGYSTPWGYFDFNRFHCHF
SPRDWQRLINNNWGFRPKRLNFKLFNIQVKEVTTNDGVTTIANNLTSTIQVFTDSEYQLPYVLGSAHQGCLPPFPA
DVFMIPQYGYLTLNNGSQAVGRSSFYCLEYFPSQMLRTGNNFQFSYTFEDVPFHSSYAHSQSLDRLMNPLIDQYLY
YLNRTQGTTSGTTNQSRLLFSQAGPQSMSLQARNWLPGPCYRQQRLSKTANDNNNSNFPWTAASKYHLNGRDS
LVNPGPAMASHKDDEEKFFPMHGNLIFGKEGTTASNAELDNVMITDEEEIRTTNPVATEQYGTVANNLQSSNTAP
TTRTVNDQGALPGMVWQDRDVYLQGPIWAKIPHTDGHFHPSPLMGGFGLKHPPPQILIKNTPVPANPSTTFSAA
KFASFITQYSTGQVSVEIEWELQKENSKRWNPEIQYTSNYYKSNNVEFAVNTEGVYSEPRPIGTRYLTRNL
(= SEQ ID NO: 1 of WO 2016/012285)
SEQ ID NO: 11
DSFSANQEIRYSEVTPYHVTSVWTKGVTPPANFTQGEDVFHAPYVANQGWYDITKTFNGKDDLLCGAATAGNML
HWWFDQNKDQIKRYLEEHPEKQKINFNGEQMFDVKEAIDTKNHQLDSKLFEYFKEKAFPYLSTKHLGVFPDHVID
MFINGYRLSLTNHGPTPVKEGSKDPRGGIFDAVFTRGDQSKLLTSRHDFKEKNLKEISDLIKKELTEGKALGLSHTYA
NVRINHVINLWGADFDSNGNLKAIYVTDSDSNASIGMKKYFVGVNSAGKVAISAKEIKEDNIGAQVLGLFTLSTGQ
DSWNQTN
(= SEQ ID NO: 2 of WO 2016/012285)
SEQ ID NO: 12
EEKTVQVQKGLPSIDSLHYLSENSKKEFKEELSKAGQESQKVKEILAKAQQADKQAQELAKMKIPEKIPMKPLHGPL
YGGYFRTWHDKTSDPTEKDKVNSMGELPKEVDLAFIFHDWTKDYSLFWKELATKHVPKLNKQGTRVIRTIPWRFL
AGGDNSGIAEDTSKYPNTPEGNKALAKAIVDEYVYKYNLDGLDVDVEHDSIPKVDKKEDTAGVERSIQVFEEIGKLIG
PKGVDKSRLFIMDSTYMADKNPLIERGAPYINLLLVQVYGSQGEKGGWEPVSNRPEKTMEERWQGYSKYIRPEQY
MIGFSFYEENAQEGNLWYDINSRKDEDKANGINTDITGTRAERYARWQPKTGGVKGGIFSYAIDRDGVAHQPKKY
AKQKEFKDATDNIFHSDYSVSKALKTVMLKDKSYDLIDEKDFPDKALREAVMAQVGTRKGDLERFNGTLRLDNPAI
QSLEGLNKFKKLAQLDLIGLSRITKLDRSVLPANMKPGKDTLETVLETYKKDNKEEPATIPPVSLKVSGLTGLKELDLSG
FDRETLAGLDAATLTSLEKVDISGNKLDLAPGTENRQIFDTMLSTISNHVGSNEQTVKFDKQKPTGHYPDTYGKTSL
RLPVANEKVDLQSQLLFGTVTNQGTLINSEADYKAYQNHKIAGRSFVDSNYHYNNFKVSYENYTVKVTDSTLGTTT
DKTLATDKEETYKVDFFSPADKTKAVHTAKVIVGDEKTMMVNLAEGATVIGGSADPVNARKVFDGQLGSETDNIS
LGWDSKQSIIFKLKEDGLIKHWRFFNDSARNPETTNKPIQEASLQIFNIKDYNLDNLLENPNKFDDEKYWITVDTY
SAQGERATAFSNTLNNITSKYWRVVFDTKGDRYSSPVVPELQILGYPLPNADTIMKTVTTAKELSQQKDKFSQKML
DELKIKEMALETSLNSKIFDVTAINANAGVLKDCIEKRQLLKK

### NUMBERED ASPECTS OF THE INVENTION

1. A method for determining neutralising antibody (NAb) titre to a viral vector comprising a capsid of interest in a sample from a subject, the method comprising a transduction inhibition assay (TIA) using a luciferase which comprises the following steps:
(a) incubating particles of a viral vector comprising the capsid of interest in (1) one or more reference solutions comprising the sample at varying dilutions, and (2) at least one control solution, wherein the viral vector of part (a) comprises a recombinant vector genome comprising a transgene encoding the luciferase;
(b) exposing each of the solutions from step (a) to a population of target cells which are susceptible to infection by the viral vector of interest;
(c) waiting for a set interval of time to allow transduction to occur;
(d) adding a substrate for the luciferase to the reference and control solutions and measuring the signal (RLU) obtained from the luciferase;
(e) comparing the signal (RLU) obtained from the luciferase in the at least one control solution with the signal (RLU) obtained from the luciferase in the reference solutions; and
(f) calculating the NAb titre;
   wherein:
(i) the set interval of time in step (c) is less than 24 hours, optionally is 19 hours or less, optionally is 12 hours or less, optionally is 8 hours or less, optionally is 6 hours or less and optionally is 3 hours; and
(ii) the luciferase is a synthetic luciferase which provides enhanced luminescence relative to a firefly luciferase.
   2. The method of aspect 1, wherein the set interval in step (c) is 6 hours or less.
   3. The method of aspect 1, wherein the set interval in step (c) is 3 hours.
   4. The method of any one of the preceding aspects wherein the synthetic luciferase has enhanced luminescence relative to a firefly luciferase having a sequence according to SEQ ID NO: 1.
   5. The method of any one of the preceding aspects wherein the synthetic luciferase is derived from a naturally-occurring wild-type luciferase having one or more of the following modifications:
(a) greater stability relative to the wild-type luciferase;
(b) smaller size (kDa) relative to the wild-type luciferase;
(c) deletion or removal of one or more subunits of the wild-type luciferase;
(d) one or more conservative or nonconservative amino acid substitutions, additions and/or deletions relative to the wild-type amino acid sequence.
   6. The method of any one of the preceding aspects wherein the synthetic luciferase is less than 50 kDa, less than 30 kDa, less than 25 kDa or less than 20 kDa.
   7. The method of any one of the preceding aspects wherein the synthetic luciferase is ATP-independent.
   8. The method of any one of the preceding aspects wherein the synthetic luciferase uses furimazine, or a derivative or variant thereof, as a substrate.
   9. The method of any one of the preceding aspects, wherein the synthetic luciferase uses coelenterazine, or a derivative or variant thereof, as a substrate.
   10. The method of any one of the preceding aspects wherein enhanced luminescence of the synthetic luciferase is determined by measuring the luminescence signal (RLU) of the synthetic luciferase and its substrate and the luminescence signal (RLU) of the firefly luciferase and its luciferine substrate under the same conditions.
   11. The method of aspect 10, wherein the signal (RLU) of the synthetic luciferase and its substrate is greater by at least 5-fold, at least 10-fold, at least 50-fold, at least 100-fold, at least 150-fold or more than the signal (RLU) of the firefly and its luciferine substrate.
   12. The method of aspect 10, wherein the signal (RLU) of the synthetic luciferase and its substrate is greater by at least 80- to 100-fold or more than the signal (RLU) of the firefly luciferase and its luciferine substrate.
   13. The method of any one of the preceding aspects wherein the synthetic bright luciferase comprises:
(a) a sequence according to SEQ ID NO: 2;
(b) a sequence having at least 90% or at least 95% identity with SEQ ID NO: 2; or
(c) a sequence which varies from SEQ ID NO: 2 by no more than one, no more than two, no more than three, no more than four or no more than five amino acids.
   14. The method of any one of aspects 1 to 12, wherein the synthetic bright luciferase comprises:
(a) a sequence according to SEQ ID NO: 3;
(b) a sequence having at least 90% or at least 95% identity with SEQ ID NO: 3; or
(c) a sequence which varies from SEQ ID NO: 3 by no more than one, no more than two, no more than three, no more than four or no more than five amino acids.
   15. The method of any one of aspects 1 to 12, wherein the synthetic bright luciferase comprises:
(a) a sequence according to SEQ ID NO: 4;
(b) a sequence having at least 90% or at least 95% identity with SEQ ID NO: 4; or
(c) a sequence which varies from SEQ ID NO: 4 by no more than one, no more than two, no more than three, no more than four or no more than five amino acids.
   16. The method of any one of the preceding aspects, wherein at least one control solution comprises a negative control solution.
   17. The method of aspect 16 wherein the negative control solution lacks antibodies to the viral vector of interest.
   18. The method of any one of aspects 1 to 15 wherein at least one control solution comprises a first negative control solution and a second positive control solution 19. The method of aspect 18 wherein the first negative control solution lacks antibodies to the viral vector of interest.
   20. The method of aspect 18 or aspect 19 wherein the second positive control solution comprises a sufficient concentration of neutralising antibodies to maximally inhibit transduction of the viral vector of interest.
   21. the method of any one of aspects 18 to 20 wherein the positive control solution comprises IVIG (in-vitro immunoglobulin).
   22. The method of aspect 21 wherein the IVIG solution has a sufficiently high concentration to ensure the maximal possible inhibition of the viral vector of interest.
   23. the method of aspect 21 or aspect 22 wherein the IVIG solution has a concentration of at least 20 μg/ml, 30 μg/ml, 50 μdml or more.
   24. The method of aspect 23 wherein the IVIG has a concentration of at least 50 μg/ml.
   25. The method of any one of aspects 18 to 24, wherein the method includes a step of serially diluting the positive control solution and carrying out steps (a) to (d) of aspect 1 on the serial dilutions in order to establish the 50% inhibition level (EC50) of the positive control solution.
   26. The method of any one of the preceding aspects wherein the sample from the patient comprises or is a plasma sample.
   27. The method of any one of the preceding aspects, wherein the population of target cells comprises (a) at least 20,000 target cells;
(b) at least 25,000 target cells;
(c) at least 50,000 target cells;
(d) at least 100,000 target cells;
(e) at least 150,000 target cells; or
(f) more than 150,000 target cells.
   28. The method of any one of the preceding aspects, wherein the population of target cells comprises mammalian cells.
   29. The method of any one of the preceding aspects wherein the target cells comprise HEK-293, HEK-293T, CHO, BHK, MDCK, 10T1/2, WEHI cells, COS, BSC 1, BSC 40, BMT 10, VERO, W138, MRCS, A549, HT1080, 293, B-50, 3T3, NIH3T3, HepG2, Saos-2, Huh7, HER, HEK, HEL, or HeLa cells.
   30. The method of any one of the preceding aspects, wherein the target cells comprise HEK293 cells or HEK293T cells.
   31. The method of any one of the preceding aspects, wherein the viral particles comprise adeno-associated virus (AAV) viral particles.
   32. The method of aspect 31, wherein the AAV particles comprise a capsid of or deriving from naturally-occurring AAV serotypes AAV1, AAV2, AAV3, AAV3B, AAV4, AAV5, AAV6, AAV7, AAV8, AAV9, AAV 10, AAV11, AAV 12, or a mixture thereof; optionally wherein the AAV particles comprise a capsid of or deriving from AAV5.
   33. The method of aspect 31, wherein the AAV particles comprise a capsid which is non-naturally occurring or synthetic or engineered.
   34. The method of aspect 33 wherein the non-naturally occurring capsid is derived from AAV3 or AAV3B.
   35. The method of aspect 33 or aspect 34 wherein the AAV particles comprise:
(a) a capsid having at least 95%, at least 96%, at least 97%, at least 98% or at least 99% identity to SEQ ID NO: 5;
(b) a capsid having at least 95%, at least 96%, at least 97%, at least 98% or at least 99% identity to SEQ ID NO: 6 (SEQ ID NO: 31 from WO 2013/029030);
(c) a capsid having at least 95%, at least 96%, at least 97%, at least 98% or at least 99% identity to SEQ ID NO: 7 (SEQ ID NO: 46 from WO 2013/029030);
(d) a capsid having at least 95%, at least 96%, at least 97%, at least 98% or at least 99% identity to SEQ ID NO: 8 (SEQ ID NO: 47 from WO 2013/029030);
(e) a capsid having at least 95%, at least 96%, at least 97%, at least 98% or at least 99% identity to SEQ ID NO: 9 (SEQ ID NO: 54 from WO 2013/029030); or
(f) a capsid having at least 95%, at least 96%, at least 97%, at least 98% or at least 99% identity to SEQ ID NO: 10 (SEQ ID NO: 56 from WO 2013/029030).
   36. The method of any one of aspects 1 to 30, wherein the viral particles comprise lentiviral particles.
   37. The method of any one of the preceding aspects, wherein step (a) of aspect 1 comprises incubating the particles of the viral vector of interest at a concentration of between 1.7×10⁷ and 1.7×10⁵ vg/ml.
   38. The method of any one of the preceding aspects, wherein step (a) of aspect 1 comprises incubating the particles of the viral vector of interest at a concentration of 8.3×10⁶ vg/ml.
   39. The method of any one of the preceding aspects, wherein step (b) of aspect 1 comprises exposing the viral particles and target cells at a ratio vg:cells (multiplicity of infection (MOI)) of 250:1 or less, 200:1 or less, 100:1 or less, 50:1 or less, 25:1 or less, 10:1 or less, or 1:1 or less.
   40. The method of any one of the preceding aspects, wherein the sample diluent comprises or is healthy human plasma.
   41. The method of any one of aspects 1 to 39 wherein the sample diluent comprises fetal bovine serum (FBS).
   42. The method of aspect 41 wherein the sample diluent comprises IgG-depleted FBS.
   43. The method of any one of aspects 40 to 42, wherein the sample diluent further comprises DMEM.
   44. The method of aspect 43 wherein the DMEM is phenol-red free DMEM.
   45. The method of any one of the preceding aspects, wherein the sample dilution and/or the positive control dilution comprises a serial dilution.
   46. The method of aspect 45 wherein the serial dilution comprises a 2-fold dilution factor.
   47. The method of aspect 45 or aspect 46 wherein the serial dilution comprises one or more of 1:2, 1:4, 1:8, 1:16, 1:32, 1:64, 1:128, 1:256, 1:1024, 1:2048, or 1:4096.
   48. The method of any one of aspects 45 to 47, wherein the serial dilution comprises one or more of 1:10, 1:50, 1:100, 1:200, 1:400, 1:800, 1:1600, or 1:3200.
   49. The method of any one of aspects 45 to 48, wherein the serial dilution comprises between 5 and 15 dilutions.
   50. The method of any one of aspects 45 to 49, wherein the serial dilution comprises between 8 and 11 dilutions.
   51. The method of any one of the preceding aspects, wherein step (b) of aspect 1 comprises providing the population of cells on an assay plate and adding the solutions thereto.
   52. The method of aspect 51 wherein the assay plate is a multi-well assay plate.
   53. The method of any one of the preceding aspects wherein step (d) of aspect 1 includes a step of lysing the cells to release the synthetic luciferase into the solution, prior to adding the substrate.
   54. The method of any one of aspects 1 to 52, wherein step (d) of aspect 1 comprises adding a substrate which can penetrate the target cells.
   55. The method of any one of aspects 1 to 52, wherein the synthetic luciferase is capable of being secreted from the target cells into the solution.
   56. The method of any one of the preceding aspects, wherein step (f) of aspect 1 comprises the step of calculating or quantifying the NAb titre as the dilution of the reference solution at which the signal (RLU) obtained is 50% of the signal (RLU) obtained in the control solution.
   57. The method of aspect 56, wherein the NAb titre is calculated or quantified as a single value (discrete titre).
   58. The method of aspect 56, wherein the NAb titre is calculated or quantified as a range of values.
   59. The method of any one of aspects 56 to 58, wherein the NAb titre is calculated or quantified through a visual comparison between the dilutions of the reference solutions and the control solution(s).
   60. The method of any one of aspects 56 to 58 wherein the NAb titre is calculated or quantified using a nonlinear regression model to fit the reference solution data to a curve and obtain a precise half-maximal value at which 50% neutralisation occurs.
   61. The method of aspect 60 wherein the non-linear regression model is applied to the luminescent signal (RLU) from each sample dilution.
   62. The method of aspect 61 wherein the non-linear regression model is applied to the luminescent signal (RLU) from each sample dilution after normalising with positive (maximal inhibition) and/or negative (0% inhibition) controls.
   63. The method of aspect 61 or aspect 62 wherein NAb titre is calculated or quantified by fitting a four-parameter variable-slope model to the luminescent signal from each sample dilution after normalising with both positive (maximal inhibition) and negative (0% inhibition) controls.
   64. The method of any one of aspects 60 to 63 wherein the NAb titre is calculated or quantified as the interpolated titre at 50% transduction inhibition (T150).
   65. An AAV viral vector which comprises or encapsidates a recombinant vector genome comprising a transgene encoding a luciferase, wherein the luciferase is a synthetic luciferase which provides enhanced luminescence relative to a firefly luciferase.
   66. The AAV viral vector of aspect 65 or the method of any one of aspects 1 to 63, wherein the recombinant vector genome is self-complementary.
   67. The AAV viral vector of aspect 65 or aspect 66, wherein the synthetic luciferase has enhanced luminescence relative to a firefly luciferase having a sequence according to SEQ ID NO: 1.
   68. The AAV viral vector of any one of aspects 65 to 67, wherein enhanced luminescence of the synthetic luciferase is determined by measuring the luminescence signal (RLU) of the synthetic luciferase and its substrate and the luminescence signal (RLU) of the firefly luciferase and its luciferine substrate under the same conditions.
   69. The AAV viral vector of any one of aspects 65 to 68, wherein the signal (RLU) of the synthetic luciferase and its substrate is greater by at least 5-fold, at least 10-fold, at least 50-fold, at least 100-fold, at least 150-fold or more than the signal (RLU) of the firefly and its luciferine substrate.
   70. The AAV viral vector of aspect 69, wherein the signal (RLU) of the synthetic luciferase and its substrate is greater by at least 80- to 100-fold or more than the signal (RLU) of the firefly luciferase and its luciferine substrate.
   71. The AAV viral vector of any one of aspects 65 to 70, wherein the synthetic bright luciferase is less than 50 kDa, less than 30 kDa, less than 25 kDa or less than 20 kDa.
   72. The AAV viral vector of any one of aspects 65 to 71, wherein the synthetic bright luciferase is ATP-independent.
   73. The AAV viral vector of any one of aspects 65 to 72, wherein the synthetic bright luciferase comprises:
(a) a sequence according to SEQ ID NO: 2;
(b) a sequence having at least 90% or at least 95% identity with SEQ ID NO: 2; or
(c) a sequence which varies from SEQ ID NO: 2 by no more than one, no more than two, no more than three, no more than four or no more than five amino acids.
   74. The AAV viral vector of any one of aspects 65 to 72, wherein the synthetic bright luciferase comprises:
(a) a sequence according to SEQ ID NO: 3;
(b) a sequence having at least 90% or at least 95% identity with SEQ ID NO: 3; or
(c) a sequence which varies from SEQ ID NO: 3 by no more than one, no more than two, no more than three, no more than four or no more than five amino acids.
   75. The AAV viral vector of any one of aspects 65 to 72, wherein the synthetic bright luciferase comprises:
(a) a sequence according to SEQ ID NO: 4;
(b) a sequence having at least 90% or at least 95% identity with SEQ ID NO: 4; or
(c) a sequence which varies from SEQ ID NO: 4 by no more than one, no more than two, no more than three, no more than four or no more than five amino acids.
   76. The AAV viral vector of any one of aspects 65 to 75, wherein the viral vector comprises:
(a) a capsid of or deriving from naturally occurring AAV serotypes AAV 3 and/or AAV3B;
(b) a capsid having at least 95%, at least 96%, at least 97%, at least 98% or at least 99% identity to SEQ ID NO: 5;
(c) a capsid having at least 95%, at least 96%, at least 97%, at least 98% or at least 99% identity to SEQ ID NO: 6 (SEQ ID NO: 31 from WO 2013/029030);
(d) a capsid having at least 95%, at least 96%, at least 97%, at least 98% or at least 99% identity to SEQ ID NO: 7 (SEQ ID NO: 46 from WO 2013/029030);
(e) a capsid having at least 95%, at least 96%, at least 97%, at least 98% or at least 99% identity to SEQ ID NO: 8 (SEQ ID NO: 47 from WO 2013/029030);
(f) a capsid having at least 95%, at least 96%, at least 97%, at least 98% or at least 99% identity to SEQ ID NO: 9 (SEQ ID NO: 54 from WO 2013/029030); or
(g) a capsid having at least 95%, at least 96%, at least 97%, at least 98% or at least 99% identity to SEQ ID NO: 10 (SEQ ID NO: 56 from WO 2013/029030).
   77. A method of determining whether a patient is eligible for gene therapy using a viral vector comprising a capsid of interest, the method comprising determining the NAb titre of the patient to said viral vector using the method of any one of aspects 1 to 64 above and comparing it with a pre-determined threshold value, wherein if the Nab titre is at or below the threshold value, the patient is eligible for gene therapy using the viral vector.
   78. A method of monitoring the progress of depletion of immunoglobulin which is specific for a viral vector comprising a capsid of interest, such as plasmapheresis or targeted (e.g. affinity-based) depletion of immunoglobulin, in a patient wherein the method comprises the steps of:
(a) determining the patient NAb titre to a viral vector comprising a capsid of interest within a set interval of time following a round of immunoglobulin depletion by carrying out the method of any one of aspects 1-64 on a sample from the patient;
(b) determining whether the NAb titre following immunoglobulin depletion is below a predetermined threshold value for eligibility of gene therapy;
and optionally
(c) determining, based on the NAb titre following immunoglobulin depletion, whether a further round of immunoglobulin depletion is appropriate.
   79. The method of aspect 78, wherein the set interval of time is 24 hours or less, 19 hours or less, 12 hours or less, 9 hours or less or 6 hours or less.
   80. The method of aspect 78 or aspect 79, wherein step (c) includes a step of comparing the NAb titre following immunoglobulin depletion with an initial NAb titre to determine whether the immunoglobulin depletion has had any significant effect.
   81. The method of any one of aspects 78 to 80, wherein the method additionally comprises:
(d) determining the patient NAb titre to a viral vector comprising a capsid of interest within a set interval of time following a further round of immunoglobulin depletion by carrying out the method of any one of aspects 1 to 64 on a sample from the patient;
(e) determining whether the NAb titre following the further round of immunoglobulin depletion is below a pre-determined threshold value for eligibility of gene therapy; and optionally
(f) determining, based on the NAb titre following immunoglobulin depletion, whether yet a further round of immunoglobulin depletion is appropriate.
   82. The method of aspect 81, wherein step (f) includes a further step of comparing the NAb titre following the previous rounds of immunoglobulin depletion with one another, and optionally with an initial NAb titre, in order to determine whether the immunoglobulin depletion has had any significant effect.
   83. The method of any one of aspects 78 to 82, wherein the method additionally comprises:
(g) determining the patient NAb titre to a viral vector comprising a capsid of interest within a set interval of time following a further round of immunoglobulin depletion by carrying out the method of the invention on a sample from the patient;
(h) determining whether the NAb titre following said further round of immunoglobulin depletion is below a pre-determined threshold value for eligibility of gene therapy; and optionally
(i) determining whether yet a further round of immunoglobulin depletion is appropriate.
   84. The method of any one of aspects 78 to 83, wherein the further round(s) of immunoglobulin depletion of step (c), step (f) and step (i) is/are carried out within 48 hours or less of the previous round of immunoglobulin depletion; optionally wherein the further round of immunoglobulin depletion of step (c), step (f) and step (i) is carried out the day after the previous round of immunoglobulin depletion; and optionally wherein the further round of immunoglobulin depletion is carried out within 24 hours of the previous round of immunoglobulin depletion.
   85. The method of any one of aspects 78 to 84, wherein the method comprises:
(a) two rounds of immunoglobulin depletion over the course of two days;
(b) three rounds of immunoglobulin depletion over the course of three days;
(c) four rounds of immunoglobulin depletion over the course of four days; or
(d) five rounds of immunoglobulin depletion over the course of five days.
   86. The method of any one of aspects 78 to 85, wherein the immunoglobulin depletion method is plasmapheresis; and optionally wherein the immunoglobulin depletion method is double filtration plasmapheresis (DFPP).
   87. The method of any one of aspects 1 to 64, wherein the method is characterised in that the population of target cells is in suspension.
   88. The method of any one of aspects 1 to 64 or aspect 87, wherein step (b) of aspect 1 comprises exposing each of the solutions from step (a) of aspect 1 to a population of target cells in suspension, which are susceptible to infection by the viral vector of interest.
   89. The method of any one of aspects 1 to 64, 87 or 88, wherein the population of target cells is not plated in advance of the method.
   90. The method of any one of aspects 1 to 64 or 87 to 89 wherein the target cells are not immobilised or otherwise adhered to an assay plate.
   91. The method of any one of aspects 1 to 64 wherein all steps of the method, including any preparation steps, are completed within 24 hours or less; optionally within 18 hours or less; optionally within 12 hours or less; optionally within 8 hours or less; and optionally in less than 8 hours.
   92. A kit comprising the AAV viral vector of any one of aspects 65 to 76, together with a reagent which includes a substrate for a luciferase encoded by the AAV viral vector.
   93. The kit of aspect 92, further comprising instructions for carrying out an assay method according to any one of aspects 1 to 64.
   94. The kit of aspect 92 or aspect 93, further comprising a container comprising target cells; optionally wherein the target cells comprise mammalian cells.
   95. The kit of aspect 94 wherein the target cells comprise HEK-293, HEK-293T, CHO, BHK, MDCK, 10T1/2, WEHI cells, COS, BSC 1, BSC 40, BMT 10, VERO, W138, MRCS, A549, HT1080, 293, B-50, 3T3, NIH3T3, HepG2, Saos-2, Huh7, HER, HEK, HEL, or HeLa cells.
   96. The kit of aspect 94, wherein the target cells comprise HEK293 cells or HEK293T cells.
   97. The kit of any one of aspects 92 to 96, wherein the kit further comprises insulating means.
   98. The kit of any one of aspects 92 to 97, wherein the kit further comprises cooling means.
   99. The kit of any one of aspects 94 to 96, wherein the container comprises insulating means.
   100. The kit of any one of aspects 94 to 96 or aspect 99, wherein the container comprises cooling means.
   101. An AAV viral vector for use in a method of treating a genetic disorder, the method comprising:
   (a) performing a method of immunoglobulin depletion on a patient;
   (b) monitoring the progress of depletion of immunoglobulin using the method of any one of aspects 78 to 86; and
   (c) administering the AAV viral vector once the immunoglobulin is sufficiently depleted, wherein the AAV viral vector comprises a transgene that encodes a polypeptide implicated in the genetic disorder, the AAV viral vector comprises a capsid, and the patient has antibodies to the capsid.
   102. The method of any one of aspects 78 to 85, wherein the immunoglobulin depletion method is:
(a) a method of immunodepletion which specifically targets immunoglobulin;
(b) a method of immunodepletion which uses an extracorporeal device which binds IgG; or
(c) a method of immunodepletion which comprises the administration of an agent or enzyme (such as a IgG cysteine protease or IgG endoglycosidases) which digests human IgG, optionally wherein the method comprises administering to the subject an agent or enzyme which reduces Fc receptor binding of serum IgG molecules; optionally wherein the agent or enzyme is an IgG cysteine protease from a Streptococcus bacterium such as Streptococcus pyogenes, or an IgG endoglycosidase from a Streptococcus bacterium, such as Streptococcus pyogenes, Streptococcus equi or Streptococcus zooepidemicus, or from Corynebacterium pseudotuberculosis, Enterococcus faecalis, or Elizabethkingia meningoseptica; and optionally wherein the agent or enzyme comprises a sequence according to SEQ ID NO: 11 or SEQ ID NO: 12, or a fragment or variant thereof which has IgG cysteine protease activity.

1. A method for determining neutralising antibody (NAb) titre to a viral vector comprising a capsid of interest in a sample from a subject, the method comprising a transduction inhibition assay (TIA) using a luciferase which includes the following steps:
(a) incubating particles of a viral vector comprising the capsid of interest in (1) one or more reference solutions comprising the sample at varying dilutions, and (2) at least one control solution, wherein the viral vector of part (a) comprises a recombinant vector genome comprising a transgene encoding the luciferase;
(b) exposing each of the solutions from step (a) to a population of target cells which are susceptible to infection by the viral vector of interest;
(c) waiting for a set interval of time to allow transduction to occur;
(d) adding a substrate for the luciferase to the reference and control solutions and measuring the signal (RLU) obtained from the luciferase;
(e) comparing the signal (RLU) obtained from the luciferase in the at least one control solution with the signal (RLU) obtained from the luciferase in the reference solutions; and
(f) calculating the NAb titre;
wherein:
(i) the set interval of time in step (c) is less than 24 hours, optionally is 19 hours or less, optionally is 12 hours or less, optionally is 8 hours or less, optionally is 6 hours or less and optionally is 3 hours; and
(ii) the luciferase is a synthetic luciferase which provides enhanced luminescence relative to a firefly luciferase.

2. The method for determining NAb titre according to claim 1, wherein the set interval in step (c) is 6 hours or less; and optionally wherein the set interval in step (c) is 3 hours.

3. The method for determining NAb titre according to any one of the preceding claims wherein the synthetic luciferase has enhanced luminescence relative to a firefly luciferase having a sequence according to SEQ ID NO: 1.

4. The method for determining NAb titre according to any one of the preceding claims wherein enhanced luminescence of the synthetic luciferase is determined by measuring the luminescence signal (RLU) of the synthetic luciferase and its substrate and the luminescence signal (RLU) of the firefly luciferase and its luciferine substrate under the same conditions.

5. The method for determining NAb titre according to claim 4, wherein the signal (RLU) of the synthetic luciferase and its substrate is greater by at least 5-fold, at least 10-fold, at least 50-fold, at least 100-fold, at least 150-fold or more than the signal (RLU) of the firefly and its luciferine substrate.

6. The method for determining NAb titre according to any one of the preceding claims wherein the synthetic bright luciferase comprises:
(a) a sequence according to SEQ ID NO: 2;
(b) a sequence having at least 90% or at least 95% identity with SEQ ID NO: 2;
(c) a sequence which varies from SEQ ID NO: 2 by no more than one, no more than two, no more than three, no more than four or no more than five amino acids;
(d) a sequence according to SEQ ID NO: 3;
(e) a sequence having at least 90% or at least 95% identity with SEQ ID NO: 3; or
(f) a sequence which varies from SEQ ID NO: 3 by no more than one, no more than two, no more than three, no more than four or no more than five amino acids;
(g) a sequence according to SEQ ID NO: 4;
(h) a sequence having at least 90% or at least 95% identity with SEQ ID NO: 4; or
(i) a sequence which varies from SEQ ID NO: 4 by no more than one, no more than two, no more than three, no more than four or no more than five amino acids.

7. The method for determining NAb titre according to any one of the preceding claims, wherein:
(a) at least one control solution comprises a negative control solution which lacks antibodies to the viral vector of interest; or
(b) at least one control solution comprises a first negative control solution which lacks antibodies to the viral vector of interest, and a second positive control solution which comprises a sufficient concentration of neutralising antibodies to maximally inhibit transduction of the viral vector.

8. The method for determining NAb titre according to claim 7(b), wherein the positive control solution comprises IVIG (in-vitro immunoglobulin); optionally wherein the IVIG has a concentration of at least 20 μg/ml, 30 μg/ml, 50 μg/ml or more.

9. The method for determining NAb titre according to any one of the preceding claims wherein the sample from the patient is a plasma sample.

10. The method for determining NAb titre according to any one of the preceding claims wherein the sample diluent comprises IgG-depleted fetal bovine serum; optionally wherein the sample diluent further comprises DMEM.

11. The method for determining NAb titre according to any one of the preceding claims wherein the population of target cells comprises at least 20,000, at least 25,000, at least 50,000, at least 100,000, at least 150,000, or more than 150,000 target cells.

12. The method for determining NAb titre according to any one of the preceding claims wherein the target cells are HEK293 or HEK293T cells.

13. The method for determining NAb titre according to any one of the preceding claims wherein the viral particles are adeno-associated virus (AAV) viral particles.

14. The method for determining NAb titre according to claim 13 wherein the viral vector comprises:
(a) a capsid of or deriving from naturally occurring AAV serotypes AAV 3 and/or AAV3B;
(b) a capsid having at least 95%, at least 96%, at least 97%, at least 98% or at least 99% identity to SEQ ID NO: 5;
(c) a capsid having at least 95%, at least 96%, at least 97%, at least 98% or at least 99% identity to SEQ ID NO: 6;
(d) a capsid having at least 95%, at least 96%, at least 97%, at least 98% or at least 99% identity to SEQ ID NO: 7;
(e) a capsid having at least 95%, at least 96%, at least 97%, at least 98% or at least 99% identity to SEQ ID NO: 8;
(f) a capsid having at least 95%, at least 96%, at least 97%, at least 98% or at least 99% identity to SEQ ID NO: 9;
(g) a capsid having at least 95%, at least 96%, at least 97%, at least 98% or at least 99% identity to SEQ ID NO: 10; or
(h) a capsid of or deriving from naturally occurring serotype AAV5.

15. The method for determining NAb titre according to any one of the preceding claims, wherein the NAb titre is calculated or quantified using a nonlinear regression model to fit the reference solution data to a curve and obtain a precise half-maximal value at which 50% neutralisation occurs.

16. An AAV vector which encapsidates or comprises a recombinant vector genome comprising a transgene encoding a luciferase, wherein the luciferase is a synthetic luciferase which provides enhanced luminescence relative to a firefly luciferase.

17. The AAV vector of claim 16, or the method of any one of claims 1 to 15, wherein the recombinant vector genome is self-complementary.

18. The AAV vector of claim 16 or claim 17, wherein the recombinant vector genome comprises a transgene encoding a synthetic bright luciferase which comprises or consists of:
(a) a sequence according to SEQ ID NO: 2;
(b) a sequence having at least 90% or at least 95% identity with SEQ ID NO: 2;
(c) a sequence which varies from SEQ ID NO: 2 by no more than one, no more than two, no more than three, no more than four or no more than five amino acids;
(d) a sequence according to SEQ ID NO: 3;
(e) a sequence having at least 90% or at least 95% identity with SEQ ID NO: 3;
(f) a sequence which varies from SEQ ID NO: 3 by no more than one, no more than two, no more than three, no more than four or no more than five amino acids;
(g) a sequence according to SEQ ID NO: 4;
(h) a sequence having at least 90% or at least 95% identity with SEQ ID NO: 4; or
(i) a sequence which varies from SEQ ID NO: 4 by no more than one, no more than two, no more than three, no more than four or no more than five amino acids.

19. The AAV vector of any one of claims 16 to 18, wherein the viral particles comprise:
(a) a capsid of or deriving from naturally occurring AAV serotypes AAV 3 and/or AAV3B;
(b) a capsid having at least 95%, at least 96%, at least 97%, at least 98% or at least 99% identity to SEQ ID NO: 5;
(c) a capsid having at least 95%, at least 96%, at least 97%, at least 98% or at least 99% identity to SEQ ID NO: 6;
(d) a capsid having at least 95%, at least 96%, at least 97%, at least 98% or at least 99% identity to SEQ ID NO: 7;
(e) a capsid having at least 95%, at least 96%, at least 97%, at least 98% or at least 99% identity to SEQ ID NO: 8;
(f) a capsid having at least 95%, at least 96%, at least 97%, at least 98% or at least 99% identity to SEQ ID NO: 9; or
(g) a capsid having at least 95%, at least 96%, at least 97%, at least 98% or at least 99% identity to SEQ ID NO: 10.

20. A method of determining whether a patient is eligible for gene therapy using a viral vector comprising a capsid of interest, the method comprising determining the NAb titre of the patient to said viral vector using the method of any one of claims 1 to 15 and comparing it with a pre-determined threshold value, wherein if the Nab titre is at or below the threshold value, the patient is eligible for gene therapy using the viral vector.

21. A method of monitoring the progress of depletion of immunoglobulin which is specific for a viral vector comprising a capsid of interest, such as plasmapheresis or targeted depletion of immunoglobulin, in a patient wherein the method comprises the steps of:
(a) determining the patient NAb titre to a viral vector comprising a capsid of interest within a set interval of time following a round of immunoglobulin depletion by carrying out the method of any one of claims 1 to 15 on a sample from the patient;
(b) determining whether the NAb titre following immunoglobulin depletion is below a predetermined threshold value for eligibility of gene therapy;
and optionally
(c) determining, based on the NAb titre following immunoglobulin depletion, whether a further round of immunoglobulin depletion is appropriate; and optionally comparing the NAb titre following immunoglobulin depletion with an initial NAb titre to determine whether the immunoglobulin depletion has had any significant effect.

22. The method of claim 21, wherein the method additionally comprises:
(d) determining the patient NAb titre to a viral vector comprising a capsid of interest within a set interval of time following a further round of immunoglobulin depletion by carrying out the method of any one of claims 1 to 24 on a sample from the patient;
(e) determining whether the NAb titre following said further round of immunoglobulin depletion is below a pre-determined threshold value for eligibility of gene therapy;
and optionally
(f) determining, based on the NAb titre following immunoglobulin depletion, whether yet a further round of immunoglobulin depletion is appropriate; and optionally comparing the NAb titre following the previous rounds of immunoglobulin depletion with one another, and optionally with an initial NAb titre, in order to determine whether the immunoglobulin depletion has had any significant effect.

23. The method of claim 21 or claim 22, wherein the method additionally comprises:
(g) determining the patient NAb titre to a viral vector comprising a capsid of interest within a set interval of time following a further round of immunoglobulin depletion by carrying out the method of the invention on a sample from the patient;
(h) determining whether the NAb titre following said further round of immunoglobulin depletion is below a pre-determined threshold value for eligibility of gene therapy;
and optionally
(i) determining whether yet a further round of immunoglobulin depletion is appropriate.

24. The method of any one of claims 21 to 23, wherein the set interval of time is 24 hours or less, 19 hours or less, 12 hours or less, 9 hours or less or 6 hours or less.

25. The method of any one of claims 21 to 24, wherein the further round(s) of immunoglobulin depletion of step (c), step (f) and step (i) is/are carried out within 48 hours or less of the previous round of immunoglobulin depletion; optionally wherein the further round of immunoglobulin depletion of step (c), step (f) and step (i) is carried out the day after the previous round of immunoglobulin depletion; and optionally wherein the further round of immunoglobulin depletion is carried out within 24 hours of the previous round of immunoglobulin depletion.

26. The method of any one of claims 21 to 25, wherein the method comprises:
(a) two rounds of immunoglobulin depletion over the course of two days;
(b) three rounds of immunoglobulin depletion over the course of three days;
(c) four rounds of immunoglobulin depletion over the course of four days; or
(d) five rounds of immunoglobulin depletion over the course of five days.

27. The method of any one of claims 21 to 26, wherein the immunoglobulin depletion method is plasmapheresis; and optionally wherein the immunoglobulin depletion method is double filtration plasmapheresis (DFPP).

28. The method of any one of claims 21 to 26, wherein the immunoglobulin depletion method is:
(a) a method of immunodepletion which specifically targets immunoglobulin;
(b) a method of immunodepletion which uses an extracorporeal device which binds IgG; or
(c) a method of immunodepletion which comprises the administration of an agent or enzyme (such as a IgG cysteine protease or IgG endoglycosidases) which digests human IgG, optionally wherein the method comprises administering to the subject an agent or enzyme which reduces Fc receptor binding of serum IgG molecules; optionally wherein the agent or enzyme is an IgG cysteine protease from a Streptococcus bacterium such as Streptococcus pyogenes, or an IgG endoglycosidase from a Streptococcus bacterium, such as Streptococcus pyogenes, Streptococcus equi or Streptococcus zooepidemicus, or from Corynebacterium pseudotuberculosis, Enterococcus faecalis, or Elizabethkingia meningoseptica; and optionally wherein the agent or enzyme comprises a sequence according to SEQ ID NO: 11 or SEQ ID NO: 12, or a fragment or variant thereof which has IgG cysteine protease activity.

29. An AAV viral vector for use in a method of treating a genetic disorder, the method comprising:
(a) performing a method of immunoglobulin depletion on a patient;
(b) monitoring the progress of depletion of immunoglobulin using the method of any one of claims 21 to 27; and
(c) administering the AAV viral vector once the immunoglobulin is sufficiently depleted, wherein the AAV viral vector comprises a transgene that encodes a polypeptide implicated in the genetic disorder, the AAV viral vector comprises a capsid, and the patient has antibodies to the capsid.
